(19) United States
(12) Patent Application Publication
(10) Pub. No.: US 2025/0388617 A1
(43) Pub. Date: Dec. 25, 2025

(54) DOUBLE-STRANDED RNA COMPRISING NUCLEOTIDE ANALOG CAPABLE OF REDUCING OFF-TARGET TOXICITY

(71) Applicant: Rona Bioscience, Limited, Hong Kong (CN)

(72) Inventors: Jinyu Huang, Shanghai (CN); Hao Zou, Shanghai (CN); Hongli Guo, Shanghai (CN)

(73) Assignee: Rona Bioscience, Limited, Hong Kong (CN)

(21) Appl. No.: 18/878,942

(22) PCT Filed: Jun. 26, 2023
(86) PCT No.: PCT/CN2023/102403

§ 371 Date: Dec. 26, 2024

(30) Foreign Application Priority Data
Jun. 27, 2022 (CN) 202210744263.8
Aug. 8, 2022 (CN) 202210948347.3
Nov. 24, 2022 (CN) 202211483699.2

(51) Int. Cl. C07H 19/173; C12N 15/113

(52) U.S. Cl. CPC C07H 19/173; C12N 15/113; C12N 2310/14

(57) ABSTRACT

The present invention provides a double-stranded RNA comprising a nucleotide analog. The double-stranded RNA of the present invention exhibits one or more of enhanced stability, reduced off-target toxicity, and enhanced effectiveness.

The present invention claims priority to the China Patent Application No. CN202210744263.8 filed on Jun. 27, 2022, to the China Patent Application No. CN202210948347.3 filed on Aug. 8, 2022, and to the China Patent Application No. CN202211483699.2 filed on Nov. 24, 2022, the disclosure of which applications are incorporated herein by reference in their entirety.

### FIELD OF THE INVENTION

The present invention relates to the technical field of medical and pharmaceutical science, and particularly relates to a double-stranded RNA comprising a nucleotide analog.

### BACKGROUND OF THE INVENTION

RNA interference is a phenomenon of specific and highly efficient degradation of the target mRNA induced by a double-stranded RNA (dsRNA). Incorporation of a heat-labile nucleotide, such as glycerol nucleic acid (GNA), in the seed region of the antisense strand of dsRNA helps improve interference efficiency and reduce off-target toxicity. See, for example, PCT Publication No. WO2018098328A1.

Therefore, there is a need in this field to develop a novel nucleotide analog that, when incorporated into dsRNA, helps reduce off-target toxicity.

### SUMMARY OF THE INVENTION

The present invention solves the aforementioned problem by providing a novel nucleotide analog.

In one aspect, the present invention provides a nucleotide dimer of formula (A):

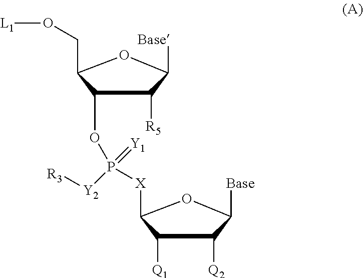

wherein each group is defined as follows.

In another aspect, the present invention provides a dsRNA molecule, or a pharmaceutically acceptable salt, tautomer, or stereoisomer thereof, comprising a sense strand and an antisense strand, each strand having 14 to 30 nucleotides; wherein the antisense strand comprises one or more nucleotide monomers of formula (III) or (IV):

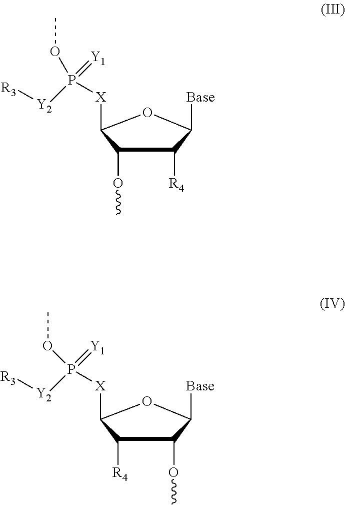

wherein the nucleotide monomers are connected from ----- to [symbol] in the 5 ′to 3′ direction: each group is defined as follows.

In another aspect, the present invention provides a nucleic acid molecule comprising in its nucleotide sequence one or more nucleotide dimers as described herein and/or nucleotide monomers as described herein.

In another aspect, the present invention provides a carrier comprising a nucleotide sequence that encodes the aforementioned dsRNA.

In another aspect, the present invention provides a cell comprising the aforementioned dsRNA or the aforementioned carrier.

In another aspect, the present invention relates to a pharmaceutical composition comprising the dsRNA molecule as described herein, and a pharmaceutically acceptable carrier or excipient.

In another aspect, the present invention relates to a kit comprising the dsRNA molecule as described herein.

In another aspect, the present invention relates to a method for inhibiting the expression of a target gene in a cell, comprising the step of introducing the dsRNA molecule as described herein into the cell.

In another aspect, the present invention relates to a method for inhibiting the expression of a target gene in a cell, comprising expressing the dsRNA molecule as described herein in the cell.

In another aspect, the present invention relates to a method for reducing off-target toxicity in a cell, comprising the step of introducing the dsRNA molecule as described herein into the cell.

In another aspect, the present invention relates to a method for reducing off-target toxicity in a cell, comprising expressing the dsRNA molecule as described herein in the cell.

Incorporation of a nucleotide of the present invention into the antisense strand of dsRNA enables the resulting dsRNA to exhibit one or more of enhanced stability, reduced off-target toxicity, and enhanced effectiveness.

### DETAILED DESCRIPTION OF THE INVENTION

### Definitions

### Chemical Definitions

Definitions of specific functional groups and chemical terms are described in more detail as follows.

When a numerical range is provided, it is intended that a particular numerical point and sub-range within said range be included. For example, “C₁₋₆ alkyl” includes alkyls C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“C₁₋₆ alkyl” refers to any straight-chain or branched hydrocarbon group being saturated and with 1 to 6 carbon atoms. In some embodiments, C₁₋₄ alkyl and C₁₋₂ alkyl are preferred. Examples of C₁₋₆ alkyl described herein include, but are not limited to: methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentyl (C₅), pentyl (C₅), neopentyl (Cₛ), 3-methyl-2-butyl (C₅), tert-pentyl (C₅) and n-hexyl (C₆). The term “C₁₋₆ alkyl” also includes any heteroalkyl in which one or more (e.g., 1, 2, 3, or 4) carbon atoms are replaced with heteroatoms (e.g., oxygen, sulfur, nitrogen, boron, silicon, phosphorus). The alkyls may be optionally substituted by one or more substituents, for example, 1 to 5 substituents, 1 to 3 substituents, or 1 substituent. The conventional abbreviations for alkyl include: Me(—CH₃), Et(—CH₂CH₃), iPr(—CH(CH₃)₂), nPr(—CH₂CH₂CH₃), n-Bu(—CH₂CH₂CH₂CH₃), or i-Bu(—CH₂CH(CH₃)₂). “C₂_alkenyl” refers to a straight-chain or branched hydrocarbon group with 2 to 6 carbon atoms and at least one carbon-carbon double bond. In some embodiments, C₂₋₄ alkenyl is preferred. Examples of C₂₋₆ alkenyl include, but are not limited to: vinyl (C₂), 1-propenyl (C₃), 2-propenyl (C₃), 1-butenyl (C₄), 2-butenyl (C₄), butadienyl (C₄), pentenyl (C₅), pentadienyl (C), and hexenyl (C₆). The term “C₂., alkenyl” also includes any heteroalkenyl in which one or more (e.g., 1, 2, 3, or 4) carbon atoms are replaced with heteroatoms (e.g., oxygen, sulfur, nitrogen, boron, silicon, phosphorus). The alkenyls may be optionally substituted by one or more substituents, for example, 1 to 5 substituents, 1 to 3 substituents, or 1 substituent. “C₂_alkynyl” refers to a straight-chain or branched hydrocarbon group with 2 to 6 carbon atoms and at least one carbon-carbon triple bond and optionally one or more carbon-carbon double bonds. In some embodiments, C₂₋₄ alkynyl is preferred. Examples of C₂0.6 alkynyl include, but are not limited to: ethynyl (C₂), 1-propynyl (C₃). 2-propvnyl(C₃), 1-butynyl (C₄), 2-butynyl (C₄), pentynyl (C₅), and hexynyl (C₆). The term “C₂. ₆ alkynyl” also includes any heteroalkynyl in which one or more (e.g., 1, 2, 3, or 4) carbon atoms are replaced with heteroatoms (e.g., oxygen, sulfur, nitrogen, boron, silicon, phosphorus). The alkynyls may be optionally substituted by one or more substituents, for example. 1 to 5 substituents, 1 to 3 substituents, or 1 substituent.

“Halo-” or “halogen” refers to (substitution by) fluorine (F), chlorine (C₁), bromine (Br), and iodine (I).

Accordingly, “C₁₋₆ haloalkyl” refers to the aforementioned “C₁₋₆ alkyl”, with one or more halogen groups. In some embodiments, a C₁₋₄ haloalkyl is particularly preferred, and a C₁0.2 haloalkyl is even more preferred. Exemplary haloalkyls include, but are not limited to: —CF₃, —CH₂F, —CHF₂, —CHFCH₂F, —CH₂CHF₂, —CF₂CF₃, —CCl₃, —CH₂C₁, —CHCl₂, and 2,2,2-trifluoro-1,1-dimethyl-ethyl. The haloalkyls may be substituted at any substitutable connection site, for example, 1 to 5 substituents, 1 to 3 substituents, or 1 substituent.

“C₁₋₆ alkoxyl” refers to an —O—R group, wherein R is as defined above for “C₁₋₆ alkyl” and “C₁₋₆ haloalkyl”.

“C₃₋₁₀cycloalkyl” refers to a non-aromatic cyclic hydrocarbon group with 3 to 10 ring carbon atoms and no heteroatoms. In some embodiments, C₄₋₇ cycloalkyl and C₃₋₆ cycloalkyl are particularly preferred, and C₅₋₆ cycloalkyl is even more preferred. A cycloalkyl herein also includes a ring system in which an aforementioned cycloalkyl ring is fused with one or more aryls or heteroaryls through any connection site(s) on the cycloalkyl ring; in this context, the number of carbons still represents the number of carbons in the cycloalkyl system. Examples of said cycloalkyls include, but are not limited to: cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), and cycloheptatrienyl (C₇). The cycloalkyls may be optionally substituted by one or more substituents, for example, 1 to 5 substituents, 1 to 3 substituents, or 1 substituent.

The term herein “3-membered to 10-membered heterocyclyl” refers to a group of a 3-membered to 10-membered non-aromatic ring system with ring carbon atom(s) and 1 to 5 ring heteroatoms, wherein each heteroatom is independently selected from nitrogen, oxygen, sulfur, boron, phosphorus and silicon. In said heterocyclyl containing one or more nitrogen atoms, the connection site may be a carbon or nitrogen atom as long as the valence permits. In some embodiments, a 4-membered to 10-membered heterocyclyl is preferred, which is a 4-membered to 10-membered non-aromatic ring system with ring carbon atom(s) and 1 to 5 ring heteroatoms; In some embodiments, a 3-membered to 8-membered heterocyclyl is preferred, which is a 3-membered to 8-membered non-aromatic ring system with ring carbon atom(s) and 1 to 4 ring heteroatoms; preferably a 3-membered to 6-membered heterocyclyl as a 3-membered to 6-membered non-aromatic ring system with ring carbon atom(s) and 1 to 3 ring heteroatoms; preferably a 4-membered to 7-membered heterocyclyl as a 4-membered to 7-membered non-aromatic ring system with ring carbon atom(s) and 1 to 3 ring heteroatoms; more preferably a 5-membered to 6-membered heterocyclyl as a 5-membered to 6-membered non-aromatic ring system with ring carbon atom(s) and 1 to 3 ring heteroatoms. A heterocyclyl herein also includes a ring system in which an aforementioned heterocyclyl ring is fused to one or more cycloalkyls through any connection site(s)on the cycloalkyl ring, or said heterocyclyl includes a ring system in which an aforementioned heterocyclyl ring is fused to one or more aryls or heteroaryls through any connection site(s) on the heterocyclyl ring; in these contexts, the number of ring members still represents the number of ring members in the heterocyclyI ring system. Exemplary 3-membered heterocyclyls containing one heteroatom include, but are not limited to: aziridinyl, oxiranyl, and thiorenyl. Exemplary 4-membered heterocyclyls containing one heteroatom include, but are not limited to: azetidinyl, oxetidinyl, and thietanyl. Exemplary 5-membered heterocyclyls containing one heteroatom include, but are not limited to: tetrahydrofuryl, dihydrofuryl, tetrahydrothienyl, dihydrothienyl, pyrrolidinyl, dihydropyrrolyl, and pyrroli-2,5-dione. Exemplary 5-membered heterocyclyls containing two heteroatoms include, but are not limited to: dioxolanyl, oxasulfuranyl, disulfuranyl, and oxazolidin-2-one. Exemplary 5-membered heterocyclyls containing three heteroatoms include, but are not limited to: triazolinyl, oxadiazolinyl and thiadiazolinyl. Exemplary 6-membered heterocyclyls containing one heteroatom include, but are not limited to: piperidinyl, tetrahydropyranyl, dihydropyridyl and thianyl. Exemplary 6-membered heterocyclyls containing two heteroatoms include, but are not limited to: piperazinyl, morpholinyl, dithianyl, and dioxanyl. Exemplary 6-membered heterocyclyls containing three heteroatoms include, but are not limited to: triazinanyl. Exemplary 7-membered heterocyclyls containing one heteroatom include, but are not limited to: azepanyl, oxepanyl, and thiepanyl. Exemplary 5-membered heterocyclyls each of which is fused with a C₆ aryl ring (also referred to herein as a 5,6-bicycloheterocyclyl) include, but are not limited to: dihydroindolyl, isodihydroindolyl, dihydrobenzofuryl, dihydrobenzothienyl, and benzoxazolinonyl. Exemplary 6-membered heterocyclyls each of which is fused with C₆ aryl ring (also referred to herein as a 6,6-bicycloheterocyclyl) include, but are not limited to: tetrahydroquinolinyl, and tetrahydroisoquinolinyl. The heterocyclyls may be optionally substituted by one or more substituents, for example, 1 to 5 substituents, 1 to 3 substituents, or I substituent.

“C₆₋₁₀ aryl” refers to a monocyclic or polycyclic (e.g., bicyclic) group that is a 4n+2 aromatic ring system having 6 to 10 ring carbon atoms and no heteroatom (e.g., with 6 or 10 x electrons shared in a cyclic arrangement).

In some embodiments, an aryl has six ring carbon atoms (“C_{G} aryl”; e.g., phenyl). In some embodiments, an aryl has ten ring carbon atoms (“C₁₀ aryl”; e.g., a naphthyl, such as I-naphthyl and 2-naphthyl). An aryl herein also includes a ring system in which an aforementioned aryl ring is fused with one or more cycloalkyls or heterocyclyls through the connection sites on said aryl ring; in this context, the number of carbon atoms still represents the number of carbon atoms in said aryl ring system. The aryls may be optionally substituted by one or more substituents, for example, 1 to 5 substituents, 1 to 3 substituents, or 1 substituent.

“5-membered to 14-membered heteroaryl” refers to a 5-membered to 14-membered monocyclic or bicyclic group of a 4n+2 aromatic ring system (e.g., with 6, 10 or 14 x electrons shared in a cyclic arrangement) that has ring carbon atom(s) and 1 to 4 ring heteroatoms, wherein each heteroatom is independently selected from nitrogen, oxygen and sulfur. In said heteroaryl containing one or more nitrogen atoms, the connection site may be a carbon or nitrogen atom as long as the valence permits. A bicyclic heteroaryl system herein may comprise one or more heteroatoms in one or both rings thereof. A heteroaryl herein also includes a ring system in which an aforementioned heteroaryl ring is fused with one or more cycloalkyls or heterocyclyls through the connection sites on said heteroaryl ring; in this context, the number of carbon atoms still represents the number of carbon atoms in said heteroaryl ring system. In some embodiments, a 5-membered to 10-membered heteroaryl is preferred, which is a 4n+2 aromatic ring system of a 5-membered to 10-membered monocyclic or bicyclic ring with ring carbon atom(s) and 1 to 4 ring heteroatoms. In some other embodiments, a 5-membered to 6-membered heteroaryl is particularly preferred, which is a 4n+2 aromatic ring system of a 5-membered to 6-membered monocyclic or bicyclic ring with ring carbon atom(s) and 1 to 4 ring heteroatoms. Exemplary 5-membered heteroaryls containing one heteroatom include, but are not limited to: pyrrolyl, furyl, and thienyl. Exemplary 5-membered heteroaryls containing two heteroatoms include, but are not limited to: imidazolyl, pyrazolyl, oxazolyl, isoxazolyl, thiazolyl, and isothiazolyl. Exemplary 5-membered heteroaryls containing three heteroatoms include, but are not limited to: triazolyl, oxadiazolyl (e.g., 1,2,4-oxadiazolyl), and thiadiazolyl. Exemplary 5-membered heteroaryls containing four heteroatoms include, but are not limited to: tetrazolyl. Exemplary 6-membered heteroaryls containing one heteroatom include, but are not limited to: pyridinyl. Exemplary 6-membered heteroaryls containing two heteroatoms include, but are not limited to: pyridazinyl, pyrimidinyl, and pyrazinyl. Exemplary 6-membered heteroaryls containing three or four heteroatoms include, but are not limited to: triazinyl, and tetrazinyl. Exemplary 7-membered heteroaryls containing one heteroatom include, but are not limited to: azepinyl, oxepinyl, and thiepinyl. Exemplary 5,6-bicycloheteroaryls include, but are not limited to: indolyl, isoindolyl, indazolyl, benzotriazolyl, benzothienyl, isobenzothienyl, benzofuryl, benzoisofuryl, benzimidazolyl, benzoxazolyl, benzisoxazolyl, benzooxadiazolyl, benzothiazolyl, benzoisothiazolyl, benzothiadiazolyl, indolizinyl, and purinyl. Exemplary 6,6-bicycloheteroaryls include, but are not limited to: naphthalidinyl, pteridinyl, quinolyl, isoquinolyl, cinnolinyl, quinoxalinyl, phthalazinyl, and quinazolinyl. The heteroaryls may be optionally substituted by one or more substituents, for example, 1 to 5 substituents, 1 to 3 substituents, or 1 substituent.

Alkyl, alkenyl, alkynyl, cycloalkyl, heterocyclyl, aryl and heteroaryl as defined herein are optionally substituted groups.

Exemplary substituents on carbon atoms include, but are not limited to: halogen, —CN, —NO₂, —N₃, —SO₂H, —SO₃H, —OH, —OR^{aa}, —ON(R^{bb})₂, —N(R^{bb})₂, —N(R^{bb})₃⁺ X—, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR*^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}—SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)N(R^{bb}), —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl, and heteroaryl, wherein each of alkyl, alkenyl, alkynyl, cycloalkyl, heterocyclyl, aryl, and heteroaryl is independently substituted with 0, 1, 2, 3, 4, or 5 R^{dd} groups;
or wherein two geminal hydrogens at a carbon atom are substituted by a group, such as ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein each of R^{aa} is independently selected from alkyl, haloalkyl, alkenyl, alkynyl, cycloalkyl, heterocyclyl, aryl and heteroaryl, or two R^{aa} groups are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups;

Each of R^{bb} is independently selected from: hydrogen, —OH, —OR^{aa}, —N(R^{cc})₂, —CN, —C(═O)R^{aa}, —C(═O)N(R^{cc})₂, —CO₂R^{cc}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are connected each other to form a heterocyclyl ring or heteroaryl ring, wherein each alkyl, alkenyl, alkynyl, cycloalkyl, heterocyclyl, aryl and heteroaryl group is independently substituted with 0, 1, 2, 3, 4 or 5 R^{dd} groups:
wherein each of R^{cc} is independently selected from hydrogen, alkyl, haloalkyl, alkenyl, alkynyl, cycloalkyl, heterocyclyl, aryl and heteroaryl, or two R^{cc} groups are connected to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{sd} groups; Each of R^{dd} is independently selected from: halogen, —CN, —NO₂, —N₃, —SO₂H, —SO₃H, —OH, —OR^{cc}, —ON(R_{ff})₂, —N(R^{ff})₂, , —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{cc}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, heteroaryl, wherein each of alkyl, alkenyl, alkynyl, cycloalkyl, heterocyclyl, aryl, and heteroaryl is independently substituted with 0, 1, 2, 3, 4, or 5 R^{gg} groups, or two geminal R^{dd} substituents may be combined to form ═O or ═S;

Each of R^{ee} is independently selected from: alkyl, haloalkyl, alkenyl, alkynyl, cycloalkyl, aryl, heterocyclyl and heteroaryl, wherein each alkyl, alkenyl, alkynyl, cycloalkyl, heterocyclyl, aryl and heteroaryl is independently substituted with 0, 1, 2, 3, 4 or 5 R^{gg} groups;
wherein each of R^{ff} is independently selected from hydrogen, alkyl, haloalkyl, alkenyl, alkynyl, cycloalkyl, heterocyclyl, aryl and heteroaryl, or two R^{ff} groups are connected each other to form a heterocyclyl ring or heteroarvl ring, wherein each alkyl, alkenyl, alkynyl, cycloalkyl, heterocyclyl, aryl and heteroaryl is independently substituted with 0, 1, 2, 3, 4 or 5 R^{gg} groups;

Each of R^{gg} is independently selected from: halogen, —CN, —NO₂, —N₃, —SO₃H, —SO₃H, —OH, —OC₁₋₆Alkyl, —ON(C₁₋₆Alkyl)₂, —N(C₁₋₆ Alkyl)₂, —N(C₁₋₆Alkyl)₃⁺X⁻, —NH(C₁₋₆Alkyl)₂⁺X⁻, —NH₂(C₁₋₆ Alkvl)⁺X⁻, —NH₃⁺X⁻, —N(OC₁₋₆Alkyl)(C₁₋₆Alkyl), —N(OH)(C₁₋₆Alkyl), —NH(OH), —SH, —SC₁₋₆Alkyl, —SS(C₁₋₆Alkyl), —C(═O)(C₁₋₆Alkyl), —CO₂H, —CO₂(C₁₋₆ Alkyl), —OC(═O)(C₁₋₆Alkyl), —OCO₂(C₁₋₆Alkyl), —C(═O)NH₂, —C(═O)N(C₁₋₆Alkyl)₂, —OC(═O)NH(C₁₋₆Alkyl), —NHC(═O)(C₁₋₆Alkyl), —N(C₁₋₆Alkyl)C(═O)(C₁₋₆ Alkyl), —NHCO₂(C₁₋₆Alkyl), —NHC(═O)N(C₁₋₆Alkyl)₂, —NHC(═O)NH(C₁₋₆Alkyl), —NHC(═O)NH₂, —C(═NH)O(C₁₋₆Alkyl), —OC(═NH)(C₁₋₆ Alkyl), —OC(═NH)OC₁₋₆Alkyl, —C(═NH)N(C₁₋₆Alkyl)₂, —C(═NH)NH(C₁₋₆Alkyl), —C(═NH)NH₂, —OC(═NH)N(C₁₋₆Alkyl)₂, —OC(NH)NH(C₁₋₆Alkyl), —OC(NH)NH₂, —NHC(NH)N(C₁₋₆Alkyl)₂, —NHC(═NH)NH₂, —NHSO₂(C₁₋₆Alkyl), —SO₂N(C₁₋₆Alkyl)₂, —SO₂NH(C₁₋₆Alkyl), —SO₂NH₂, —SO₂C₁₋₆Alkyl, —SO₂OC₁₋₆Alkyl, —OSO₂C₁₋₆Alkyl, —SOC₁₋₆Alkyl, —Si(C₁₋₆Alkyl)₃, —OSi(C₁₋₆Alkyl)₃, —C(═S)N(C₁₋₆Alkyl)₂, C(═S)NH(C₁₋₆Alkyl), C(═S)NH2, —C(═O)S(C₁₋₆Alkyl), —C(═S)SC₁₋₆Alkyl, —SC(═S)SC₁₋₆Alkyl, —P(═O)₂(C₁₋₆Alkyl), —P(═O)(C₁₋₆ Alkyl)₂, —OP(═O)(C₁₋₆Alkyl)₂, —OP(═O)(OC₁₋₆Alkyl), C₁₋₆Alkyl, C₁₋₆Haloalkyl, C₂-C₆Alkenyl, C₂-C₆Alkynyl. C₃-C₇Cycloalkyl, C₆-C₁₀Aryl, C₃-C₇Heterocyclyl, and C₅-C₁₀Heteroarly; or two geminal R^{gg} substituents may be connected to form ═O or ═S; wherein, X—is a counterion.

Exemplary substituents on a nitrogen atom include, but are not limited to: hydrogen, —OH, —OR^{aa}, —N(R^{cc})₂, —CN, —C(═O)R^{aa}, —C(═))N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two Rec groups connecting to said nitrogen atom are connected to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described above.

Some Additional Definitions

The term “siRNA” herein is a class of dsRNA molecules each of which can mediate the silencing of target RNA (e.g., mRNA, e.g., transcript of a gene encoding a protein) complementary thereto. A siRNAs is generally double-stranded, including an antisense strand complementary to the target RNA thereof and a sense strand complementary to this antisense strand. For the sake of convenience, such an mRNA is also referred to herein as mRNA to be silenced, and such a gene is also called target gene. Usually, an RNA to be silenced herein is an endogenous gene or a pathogen gene. In addition, RNAs other than mRNA (e.g., tRNA) as well as viral RNA may also be targeted.

The term “antisense strand” herein refers to a strand of a siRNA, wherein said strand contains a region that is completely, sufficiently or substantially complementary to the target sequence thereof. The term “sense strand” herein refers to a strand of a siRNA, wherein said strand contains a region that completely, sufficiently or substantially complementary to a region of an antisense strand as defined herein.

The term “complementary region” herein refers to a region on an antisense strand that is completely, sufficiently or substantially complementary to the target mRNA sequence thereof. In cases where a complementary region is incompletely complementary to the target sequence thereof, a mismatch may be located in an internal or terminal region of the molecule. Typically, a mismatch most tolerant is located in a terminal region, e.g., within 5,4,3, 2 or 1 nucleotide at the 5′ and/or 3′ end. A region in an antisense strand, which is most sensitive to mismatch, is called “seed region”. For example, in a siRNA containing a strand of 19 nt, the 19^{th} site (counting from the 5′ end to the 3′ end) can tolerate some mismatches.

The term “complementary” refers to the ability of a first polynucleotide to hybridize with a second polynucleotide under certain conditions, such as stringent conditions. For example, stringent conditions may include 400 mM NaCl, 40 mM PIPES pH 6.4, 1 mM EDTA, and 50° C. or 70° C. for 12-16 hours. With respect to fulfilling the above required capabilities related to the hybridization ability thereof, said “complementary” sequences may also include or be entirely composed of non-Watson-Crick base pairs and/or base pairs formed from non-natural as well as modified nucleotides. Such non-Watson-Crick base pairs include, but are not limited to, G:U wobble base pairing or Hoogsteen base pairing.

A polynucleotide that is “at least partially complementary”, “sufficiently complementary” or “substantially complementary” to a messenger RNA (mRNA) refers to a polynucleotide that is substantially complementary to a continuous portion of the mRNA of interest. For example, a polynucleotide is at least partially complementary to an mRNA encoding PCSK9, when the sequence thereof is substantially complementary to an uninterrupted portion of said PCSK9 mRNA. The terms “complementary,” “completely complementary,” “sufficiently complementary” and “substantially complementary” as used herein may be applied to base pairing between the sense strand and antisense strand of a siRNA, or between the antisense strand of a siRNA reagent and the target sequence thereof.

“Sufficiently complementary” refers to the extent to which the sense strand only needs to be complementary to the antisense strand to maintain the overall double-stranded character of the molecule. In other words, although perfect complementarity is generally desired, in some cases, particularly in the antisense strand, one or more, e.g., 6, 5, 4, 3, 2, or 1 mismatch (relative to the target mRNA) may be included, but the sense and antisense strands can still maintain the overall double-stranded character of the molecule.

The term “shRNA” herein refers to short hairpin RNA. An shRNA comprises two short inverted repeat sequences. An shRNA cloned into an shRNA expression vector comprises two short inverted repeat sequences, separated by a loop sequence, forming a hairpin structure and controlled by the RNA polymerase III (pol Il1) promoter. Subsequently, 5 to 6 Ts are ligated as transcription terminators of pol III.

“Nucleoside” is a compound comprising two substances; one is a purine base or a pyrimidine base, and the other is a ribose or a deoxyribose; “nucleotide” is a compound comprising three substances: one is a purine base or a pyrimidine base, another is a ribose or deoxyribose, and the third is a phosphoric acid; and “oligonucleotide” refers to, for example, a nucleic acid molecule (RNA or DNA) with a length of less than 100, 200, 300 or 400 nucleotides.

The term “base” is a fundamental building block of nucleosides, nucleotides and nucleic acids; as always containing nitrogen, said base is also referred to as “nitrogenous base.” Unless otherwise specified, the capital letters herein, i.e., A. U, T, G and C, denote the bases of nucleotides, which is adenine, uracil, thymine, guanine and cytosine, respectively.

As used herein, the “modification” of nucleotides includes, but is not limited to: methoxyl substitution (methoxy-modified), fluorine substitution (fluoro-modified), connection with a phosphorothioate group, or protection with a conventional protecting group. For example, a fluoro-modified nucleotide refers to a nucleotide formed by substituting the hydroxyl at the 2′ position of the ribosyl of the nucleotide with a fluorine atom, while a methoxy-modified nucleotide refers to a nucleotide formed by substituting the 2′-hydroxyl of the ribosyl with a methoxyl.

“Modified nucleotides” herein include, but are not limited to: a 2′-O-methyl modified nucleotide, a 2′-fluoro modified nucleotide, a 2′-deoxy-modified nucleotide, an inosine ribonucleotide, an abasic nucleotide, an inverted abasic deoxyribonucleotide, a nucleotide comprising a phosphorothioate group, a nucleotide modified by vinylphosphonate, a locked nucleotide, 2′-amino-modified nucleotide, 2′-alkyl-modified nucleotide, a morpholino nucleotide, a phosphoramidate, a non-natural base comprising nucleotide, and a terminal nucleotide linked to a cholesteryl derivative or a dodecanoic acid bisdecylamide group, deoxyribonucleotide, or a nucleotide with protection of a conventional protecting group. For example, a 2-fluoro modified nucleotide refers to a nucleotide formed by substituting the hydroxyl at the 2′ position of the ribosyl in a nucleotide with a fluorine atom. Said 2′-deoxy-modified nucleotide refers to a nucleotide formed by substituting the 2′-hydroxyl of the ribosvl with a methoxvl.

The term “reactive phosphorus group” refers to a phosphorus-containing group included within a nucleotide unit or a nucleotide analogue unit, wherein the group can undergo a nucleophilic attack to react with a hydroxyl or amine group in another molecule, especially another nucleotide unit or nucleotide analogue unit. Typically, such a reaction generates an ester-type internucleoside bond connecting a said first nucleotide unit or a said first nucleotide analogue unit with a said second nucleotide unit or a said second nucleotide analogue unit. A reactive phosphorus group can be selected from phosphoramidite, H-phosphonate, alkyl-phosphonate, phosphate or phosphate mimics, including but not limited to: natural phosphate, phosphorothioate, phosphorodithioate, borano phosphate, borano thiophosphate, phosphonate, halogen substituted phosphonates and phosphates, phosphoramidates, phosphodiester, phosphotriester, thiophosphodiester, thiophosphotriester, diphosphates and triphosphates, preferably P(OCH2CH₂CNXN(iPr)₂).

“Protecting group” refers to any atom or group of atoms added to a molecule to prevent undesired chemical reactions of existing groups within the molecule. A “protecting group” may be an unstable chemical moiety known in the art, which is used to protect reactive groups such as hydroxyl, amino and thiol groups to prevent undesired or premature reactions during chemical synthesis. Protecting groups are typically used selectively and/or orthogonally to protect sites during the reactions of other reactive sites, which can then be removed to leave the unprotected groups intact or available for further reactions.

A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Preferred protecting groups are selected from acetyl (Ac), benzoyl (Bzl), benzyI (Bn), isobutyryl (iBu), phenylacetyl, benzyloxymethyl acetal (BOM), beta-methoxyethoxymethyl ether (MEM), methoxymethylether (MOM), p-methoxybenzyl ether (PMB), methylthiomethyl ether, pivaloyl (Piv), tetrahydropyranyl (THP), triphenylmethyl (Trt), methoxytrityl [(4-methoxyohenyl)diphenylmethyl-](MMT), dimethoxytrityl, ibis-(4-methoxyphenyl)phenylmethyl (DMT), trimethylsilyl ether (TMS), tert-butyldimethylsilyl ether (TBDMS), tri-iso-propylsilyloxymethyl ether (TOM), tri-isopropylsilyl ether (TIPS), methyl ethers, ethoxyethyl ethers (EE) N,N-dimethylformamidine and 2-cynaonethyl (CE).

“Hydroxy-protecting group” refers to a group that can prevent a hydroxyl from undergoing chemical reactions and can be removed under specific conditions to restore the hydroxyl. The main hydroxy-protecting groups include silane-type, acyl-type or ether-type protecting groups, preferably the following:
trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

As used herein, the term “pharmaceutically acceptable salt” represents carboxylates or amino acid salts of a compound of the present invention, which are suitable for contact with patient tissues within the scope of sound medical judgment without causing excessive toxicity, irritation, allergic reactions, etc., and are effective in terms of the intended use with a reasonable benefit/risk ratio, said salt includes, where applicable, the zwitterionic form of a compound of the present invention.

The present invention includes tautomers, which are functional-group isomers resulting from the rapid migration of an atom in a molecule between two positions. A compound with different tautomeric forms, said herein, refers to all the tautomers and does not be restricted to any specific tautomeric form.

A compound of the present invention may include one or more asymmetric centers, and thus may exist in various stereoisomeric forms, such as enantiomers and/or diastereomers. For example, a compound of the present invention may be one of the forms of enantiomer, diastereoisomer or geometric isomer (e.g. a cis isomer or a trans isomer), or may be a mixture of any type of stereoisomerism, including a racemic mixture and a mixture enriched with one or more forms of stereoisomer. An isomer herein may be achieved by separating from a mixture via any method known to those skilled in the art, wherein the method includes chiral high-pressure liquid chromatography (HPLC) and the formation and crystallization of chiral salts; alternatively, a preferred isomer may be prepared through asymmetric synthesis.

The present invention also includes isotopically labeled compounds (isotopic variants) which are equivalent to those described by formula (I), except that one or more atoms are replaced with atoms with an atomic mass or mass number different from that common in nature. Examples of isotopes which may be incorporated into a compound of the present invention include isotopes of hydrogen, carbon, nitrogen, oxygen, phosphorus, sulfur, fluorine, and chlorine, such as ²H, ³H, ¹³C, ¹¹C, ¹⁴C. ¹⁵N, ¹⁸O, ¹⁷, ³¹P, ³²P, ³⁵S, ¹⁸F, and ³⁶C. Any compounds based on a compound of the present invention and containing any aforementioned isotope and/or any isotope of other atoms, the prodrugs thereof and the pharmaceutically acceptable salts of said compounds or said prodrugs all fall in the scope of the present invention. Certain isotopically labeled compounds of the present invention, such as a compound into which any radioisotope (e.g., ³H and ¹⁴C) is introduced, may be used for distribution determinations of a drug and/or the substrate tissue thereof. Tritium. i.e. ³H and carbon-14, i.e. ¹⁴C isotopes are particularly preferred, because they can be easily prepared and detected. Furthermore, substitution with an isotope heavier, such as deuterium, i.e. ²H, may in some cases be preferred because resultant increased metabolic stability may provide therapeutic benefits such as prolonged in vivo half-life or reduced dosage. An isotopically labeled compound of formula (I) of the present invention and the prodrug thereof may generally be prepared with any readily available isotopically labeled reagent instead of any non-isotopically labeled reagent, in a procedure described below and/or in a process disclosed in any of the Examples and Preparations.

Compounds of the Present Invention

The present invention particularly relates to a nucleotide dimer of formula (A):

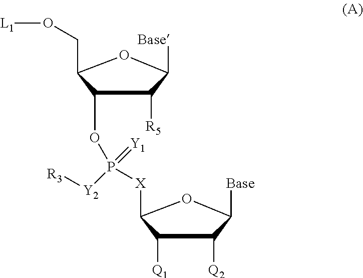

wherein one of Q₁ and Q₂ is R₄, and the other is O-L₂; L₁ is H or P₁, or a bond to the phosphorus atom P of the phosphate group at the 2 or 3′ end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂:
X is selected from —(CR₁R₂)ₘ—or —CR₁=CR₂—;
Y₁ is O, S, or NR:
Y₂ is O, S. or a bond;
R₁ and R₂ are independently selected from H, D, halogen, OH, CN, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₁₋₁₀ aryl, or 5-membered to 14-membered heteroaryl, which are optionally substituted with 1, 2, 3, 4, 5, 6, 7, 8, or more R′;
R₃ is selected from H. C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl, which are optionally substituted with 1, 2, 3, 4, 5, 6, 7, 8, or more R;
R₄ and R₅ are independently selected from H, D, OH, halogen, C₁₋₆ alkyl, C₁₋₆ haloalkyl, or C₁₋₆ alkoxyl, preferably H, F. or OMe.
P₁ is a hydroxy-protecting group, such as trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), 4,4′-dimethoxytrityl, —P(OCH₂CH₂CN)(N(iPr)₂), or —C(O)CH₂CH₂C(O)OH, preferably DMTr;
P₂ is a reactive phosphorus group, such as phosphoramidite, H-phosphonate, alkyl-phosphonate, phosphate or phosphate mimics, including but not limited to: natural phosphate, phosphorothioate, phosphorodithioate, borano phosphate, borano thiophosphate, phosphonate, halogen substituted phosphonates and phosphates, phosphoramidates, phosphodiester, phosphotriester, thiophosphodiester, thiophosphotriester, diphosphates or triphosphates, preferably P(OCH₂CH₂CN)(N(iPr)₂);
Base and Base′ are independently selected from H, a modified or unmodified base or leaving group, preferably modified or unmodified A. U, T, G, and C;
R is selected from H, C₁₋₆ alkyl, or C₁₋₆ haloalkyl;
R′ is selected from D, halogen, CN, C₁₋₆alkyl, C₁₋₆ haloalkyl, C₁₋₆ alkenyl, C₂₋₆ alkynyl, C₃₋₁₀ cycloalkyl, 3-membered to 10-membered heterocyclyl, C₆₋₁₀ aryl, 5-membered to 14-membered heteroaryl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —S(O)ⁿR^{a}, —S(O)ₙOR^{a}, —S(O)ₙNR^{a}R^{b}, —O—S(O)ₙR^{b}, —NR^{a}R^{b}, —NR^{a}C(O)R^{b}, —NR^{a}—C(O)OR^{b}, —NR^{a}—S(O)ₙR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl; or R^{a} and R^{b} and the nitrogen atom to which they are attached form a 3-membered to 10-membered heterocyclyl;
m is selected from 1, 2, 3, 4 or 5;
n is independently selected from 1 or 2.

The present invention further relates to a dsRNA molecule, or a pharmaceutically acceptable salt, tautomer, or stereoisomer thereof, comprising a sense strand and an antisense strand, each strand having 14 to 30 nucleotides; wherein the antisense strand comprises one or more nucleotide monomers of formula (III) or (IV):

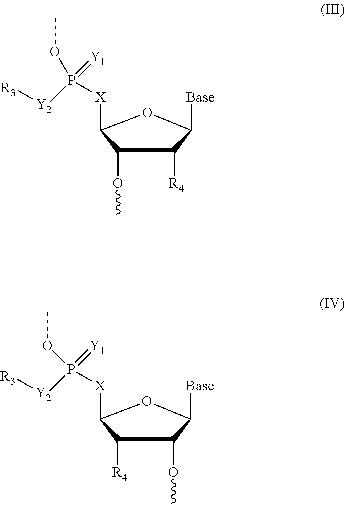

wherein the nucleotide monomers are connected from ----- to [symbol] in the 5′ to 3′ direction; each group is as defined in the context;
preferably, the nucleotide monomers are selected from:

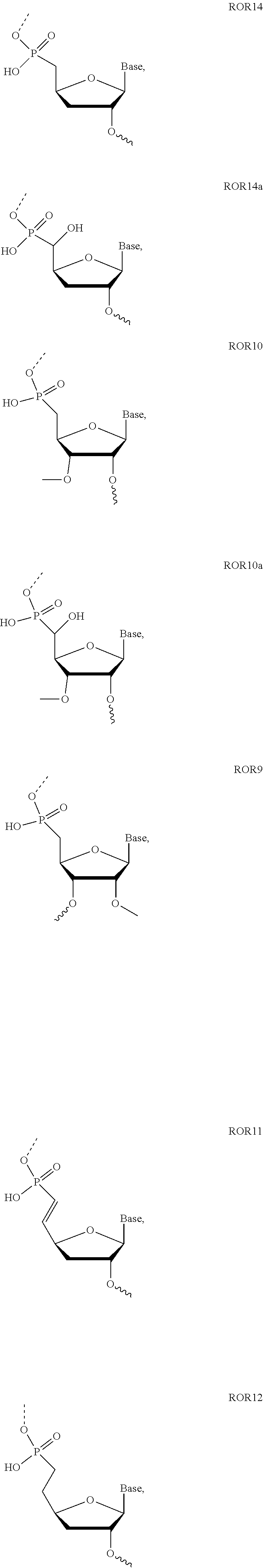

wherein Base is selected from

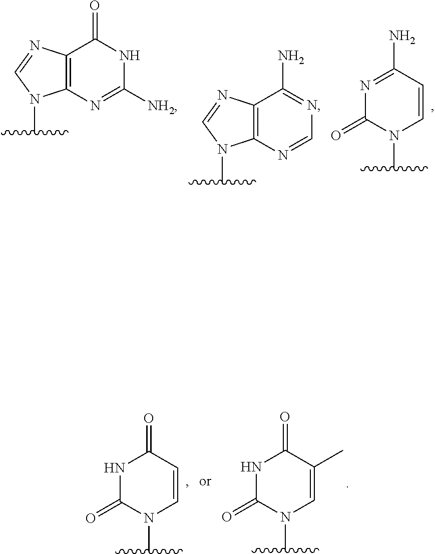

Q₁ and Q₂

In one embodiment, Q₁ is R₄, and Q₂ is O-L₂; In another embodiment, Q₁ is O-L₂, and Q₂ is R₄.

X

In one embodiment, X is —(CR₁R₂).—; In another embodiment, X is —CRI=CR₂—.

In a more specific embodiment. X is —CH₂—; In another more specific embodiment, X is —CH(OH)—; In another more specific embodiment. X is —CH₂—CH₂—; In another more specific embodiment, X is —CH═CH—.

Y₁ and Y₂

In one embodiment, Y₁ is O; In another embodiment, Y₁ is S; In another embodiment, Y₁ is NR.

In one embodiment, Y₂ is O; In another embodiment, Y is S; In another embodiment, Y₂ is a bond.

L₁ and L₂

In one embodiment, L₁ is H; In another embodiment, L₁ is P₁; In another embodiment, L₁ is a bond to the phosphorus atom P of the phosphate group at the 2′ or 3′ end of the ribose of another nucleotide or oligonucleotide.

In one embodiment, L₂ is H; In another embodiment, L₂ is P₂; In another embodiment. L₂ is a bond to the phosphorus atom P of the phosphate group at the 5′ end of the ribose of another nucleotide or oligonucleotide.

R₁ and R₂

In one embodiment, R₁ is H; In another embodiment, R₁ is D; In another embodiment, R₁ is halogen; In another embodiment, R₁ is OH; In another embodiment, R₁ is CN; In another embodiment. R₁ is C₁₋₆ alkyl; In another embodiment, R₁ is C₁₋₄ alkyl; In another embodiment. R₁ is C₁₋₆ haloalkyl; In another embodiment. R₁ is C₁₋₄ haloalkyl; In another embodiment, R₁ is C₂₋₆ alkenyl; In another embodiment, R₁ is C₂₋₆ alkynyl; In another embodiment, R₁ is C₃₋₁₀ cycloalkyl; In another embodiment, R₁ is a 3-membered to 10-membered heterocyclyl;

In another embodiment, R₁ is C₁₋₁₀ aryl; In another embodiment, R₁ is a 5-membered to 14-membered heteroaryl.

In one embodiment, R₁ is unsubstituted; In another embodiment, R₁ is substituted with one R′; In another embodiment, R₁ is substituted with two R′; In another embodiment, R₁ is substituted with three R′; In another embodiment, R₁ is substituted with four R; In another embodiment, R₁ is substituted with five R′; In another embodiment. R₁ is substituted with six R′; In another embodiment, R₁ is substituted with seven R; In another embodiment, R₁ is substituted with eight R′; In another embodiment, R₁ is substituted with a plurality of R′.

In one embodiment, R₂ is H; In another embodiment, R₂ is D; In another embodiment, R₂ is halogen; In another embodiment, R; is OH; In another embodiment, R₂ is CN; In another embodiment, R₂ is C₁₋₆ alkyl; In another embodiment, R₂ is C-₄ alkyl; In another embodiment, R₂ is C₁₋₆ haloalkyl; In another embodiment, R₂ is C₄ haloalkyl; in another embodiment. R₂ is C₂0.6 alkenyl; in another embodiment, R₂ is C₂0.6 alkynyl; In another embodiment, R₂ is C₃₋₁₀ cycloalkyl; In another embodiment, R₂ is a 3-membered to 10-membered heterocyclyl; In another embodiment. R₂ is C₆₋₁₀ aryl; In another embodiment, R₂ is a 5-membered to 14-membered heteroaryl.

In one embodiment, R₂ is unsubstituted; In another embodiment, R₂ is substituted with one R; In another embodiment. R₂ is substituted with two R′; In another embodiment, R₂ is substituted with three R′; In another embodiment, R₂ is substituted with four R′; In another embodiment, R₂ is substituted with five R′; In another embodiment, R₂ is substituted with six R′; In another embodiment, R₂ is substituted with seven R′; In another embodiment, R₂ is substituted with eight R′; In another embodiment, R₂ is substituted with a plurality of R′.

R₃

In one embodiment, R₃ is H; In another embodiment, R₃ is C₁₆ alkyl; In another embodiment, R₃ is C₁₄ alkyl, such as Me; In another embodiment. R₃ is C₁₋₆ haloalkyl; In another embodiment, R₃ is C₁₋₄ haloalkyl; In another embodiment, R₃ is C₂₋₆ alkenyl; In another embodiment, R₃ is C₂₋₆ alkynyl; In another embodiment, R₃ is C₃₋₁₀ cycloalkyl; In another embodiment, R₃ is a 3-membered to 10-membered heterocyclyl; In another embodiment, R; is C₆.,o aryl; In another embodiment, R₃ is a 5-membered to 14-membered heteroaryl.

In one embodiment, R₃ is unsubstituted; In another embodiment, R₃ is substituted with one R′; In another embodiment. R₃ is substituted with two R′; In another embodiment, R₃ is substituted with three R′; In another embodiment, R₃ is substituted with four R; In another embodiment, R₃ is substituted with five R′; In another embodiment, R₃ is substituted with six R′; In another embodiment. R₃ is substituted with seven R′; In another embodiment. R₃ is substituted with eight R′; In another embodiment, R₃ is substituted with a plurality of R′.

R₄ and R₅

In one embodiment, R₄ is H; In another embodiment, R₄ is D; In another embodiment, R₄ is OH; In another embodiment, R₄ is halogen; In another embodiment, R₄ is C₁₋₆ alkyl; In another embodiment, R₄ is C₁₋₄ alkyl; In another embodiment, R₄ is C₁₋₆ haloalkyl; In another embodiment, R₄ is C₁₋₄ haloalkyl; In another embodiment. R₄ is C₁₋₆ alkoxyl; In another embodiment, R₄ is C₁₋₄ alkoxyl, such as OMe; In another embodiment, R₄ is C₁₋₆ haloalkoxyl; In another embodiment, R₄ is C₁₋₄ haloalkoxyl.

In one embodiment, R₅ is H; In another embodiment, R₅ is D; In another embodiment, R₅ is OH; In another embodiment, R₅ is halogen, such as F; In another embodiment, R₅ is C₁₋₆ alkyl; In another embodiment, R₅ is C₁₋₄ alkyl; In another embodiment, R₅ is C₁₋₆ haloalkyl; In another embodiment, R₅ is C₁₋₄ haloalkyl; In another embodiment, R₅ is C₁₋₆ alkoxyl; In another embodiment, R₅ is C₁₋₄ alkoxyl, such as OMe; In another embodiment, R₅ is C₁₋₆ haloalkoxyl; In another embodiment. R₅ is C₁₋₄ haloalkoxyl.

P₁ and P₂

In one embodiment, P₁ is a protecting group; In another embodiment, P₁ is a hydroxy-protecting group, such as trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), 4,4′-dimethoxytrityl, —P(OCH₂CH₂CN)(N(iPr)₂), or —C(O)CH₂CH₂C(O)OH, preferably DMTr.

In one embodiment, P₂ is a reactive phosphorus group, such as phosphoramidite, H-phosphonate, alkyl-phosphonate, phosphate or phosphate mimics, including but not limited to: natural phosphate, phosphorothioate, phosphorodithioate, borano phosphate, borano thiophosphate, phosphonate, halogen substituted phosphonates and phosphates, phosphoramidates, phosphodiester, phosphotriester, thiophosphodiester, thiophosphotriester, diphosphates or triphosphates, preferably P(OCH₂CH₂CN)(N(iPr)₂).

Base and Base′

In one embodiment, Base is H; In another embodiment, Base is a modified or unmodified base or leaving group, for example, preferably modified or unmodified A, U, T, G, and C.

In one embodiment, Base′ is H, In another embodiment, Base′ is a modified or unmodified base or leaving group, for example, preferably modified or unmodified A, U, T, G, and C.

In a more specific embodiment, Base is

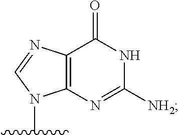 [SMILES: *n1cnc2c(=O)[nH]c(N)nc21]

In another more specific embodiment, Base is

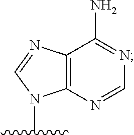 [SMILES: *n1cnc2c(N)ncnc21]

In another more specific embodiment, Base is

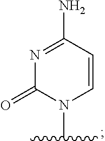 [SMILES: *n1ccc(N)nc1=O]

In another more specific embodiment, Base is

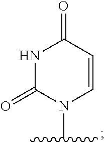 [SMILES: *n1ccc(=O)[nH]c1=O]

In another more specific embodiment, Base is

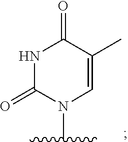 [SMILES: *n1cc(C)c(=O)[nH]c1=O]

In another more specific embodiment, Base is

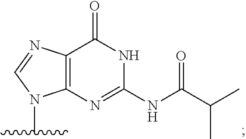 [SMILES: *n1cnc2c(=O)[nH]c(NC(=O)C(C)C)nc21]

In another more specific embodiment, Base is

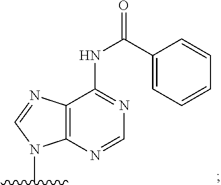 [SMILES: *n1cnc2c(NC(=O)c3ccccc3)ncnc21]

In another more specific embodiment, Base is

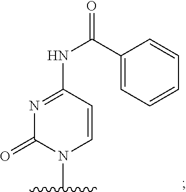 [SMILES: *n1ccc(NC(=O)c2ccccc2)nc1=O]

In another more specific embodiment. Base is

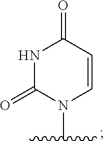 [SMILES: *n1ccc(=O)[nH]c1=O]

In another more specific embodiment, Base is

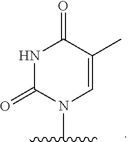 [SMILES: *n1cc(C)c(=O)[nH]c1=O]

In a more specific embodiment. Base′ is

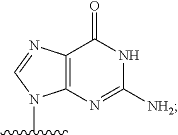 [SMILES: *n1cnc2c(=O)[nH]c(N)nc21]

In another more specific embodiment. Base′ is

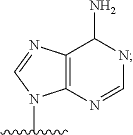

In another more specific embodiment, Base′ is

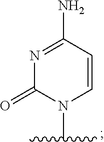 [SMILES: *n1ccc(N)nc1=O]

In another more specific embodiment,

Base′ is

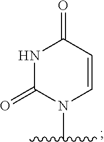 [SMILES: *n1ccc(=O)[nH]c1=O]

In another more specific embodiment. Base

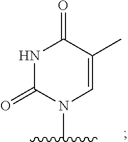 [SMILES: *n1cc(C)c(=O)[nH]c1=O]

In another more specific embodiment, Base′ is

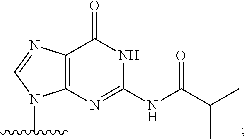 [SMILES: *n1cnc2c(=O)[nH]c(NC(=O)C(C)C)nc21]

In another more specific embodiment, Base′ is

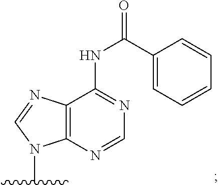 [SMILES: *n1cnc2c(NC(=O)c3ccccc3)ncnc21]

In another more specific embodiment, Base′ is

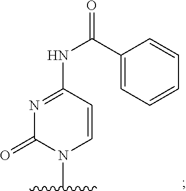 [SMILES: *n1ccc(NC(=O)c2ccccc2)nc1=O]

In another more specific embodiment, Base′ i

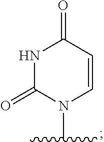 [SMILES: *n1ccc(=O)[nH]c1=O]

In another more specific embodiment, Base′ is

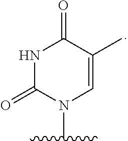 [SMILES: *n1cc(C)c(=O)[nH]c1=O]

R

In one embodiment, R is H; In another embodiment, R is C₁₋₆ alkyl; In another embodiment, R is C₁₋₆ haloalkyl.

R′

In one embodiment, R′ is D; In another embodiment, R′ is halogen; In another embodiment, R′ is CN; In another embodiment, R′ is C₁₋₆ alkyl; In another embodiment. R′ is C₁₋₆ haloalkyl; In another embodiment. R′ is C₂₋₆ alkenyl; In another embodiment. R′ is C₂₋₆ alkynyl; In another embodiment, R′ is C₃₋₁₀ cycloalkyl; In another embodiment. R′ is a 3-membered to 10-membered heterocychl; In another embodiment, R′ is C₆₋₁₀ aryl; In another embodiment, R′ is a 5-membered to 14-membered heteroaryl; In another embodiment, R′ is —OR^{a}, such as OH; In another embodiment, R′ is —OC(O)R^{a}; In another embodiment, R′ is —O—C(O)OR^{b}; In another embodiment, R′ is —O—C(O)NR^{a}R^{b}; In another embodiment, R′ is —C(O)R^{a}; In another embodiment, R′ is —C(O)OR^{a}; In another embodiment, R′ is —C(O)NR^{a}R^{b}; In another embodiment, R′ is —S(O)ₙR^{a}; In another embodiment, R′ is —S(O)ₙOR^{a}; In another embodiment, R′ is —S(O)nNR^{a}R^{b}; In another embodiment, R′ is —O—S(O)ₙR^{b}; In another embodiment, R′ is —NR^{a}R^{b}; In another embodiment, R′ is —NR^{a}C(O)R^{b}; In another embodiment. R′ is —NR^{a}—C(O)OR^{b}; In another embodiment, R′ is —NR^{a}—S(O)ₙR^{b}; In another embodiment, R′ is —NR^{a}C(O)NR^{a}R^{b}.
wherein each R^{a} is independently selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl: or R^{a} and R^{b} and the nitrogen atom to which they are attached form a 3-membered to 10-membered heterocyclyl;
each R^{b} is independently selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl; or R^{a} and R^{b} and the nitrogen atom to which they are attached fonn a 3-membered to 10-membered heterocyclyl.

m

In one embodiment, m is 0; In another embodiment, m is 1 In another embodiment, m is 2; In another embodiment, m is 3; In another embodiment, m is 4; In another embodiment, m is 5.

n

In one embodiment, n is 0; In another embodiment, n is 1 In another embodiment, n is 2; In another embodiment, n is 3; in another embodiment, n is 4; In another embodiment, n is 5.

GalNAc

In one embodiment, GalNAc is a conjugation group of formula (X):

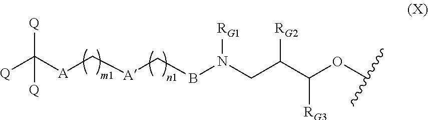

wherein,

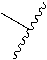

represents the position of attachment to biomolecules;
Q is independently H,

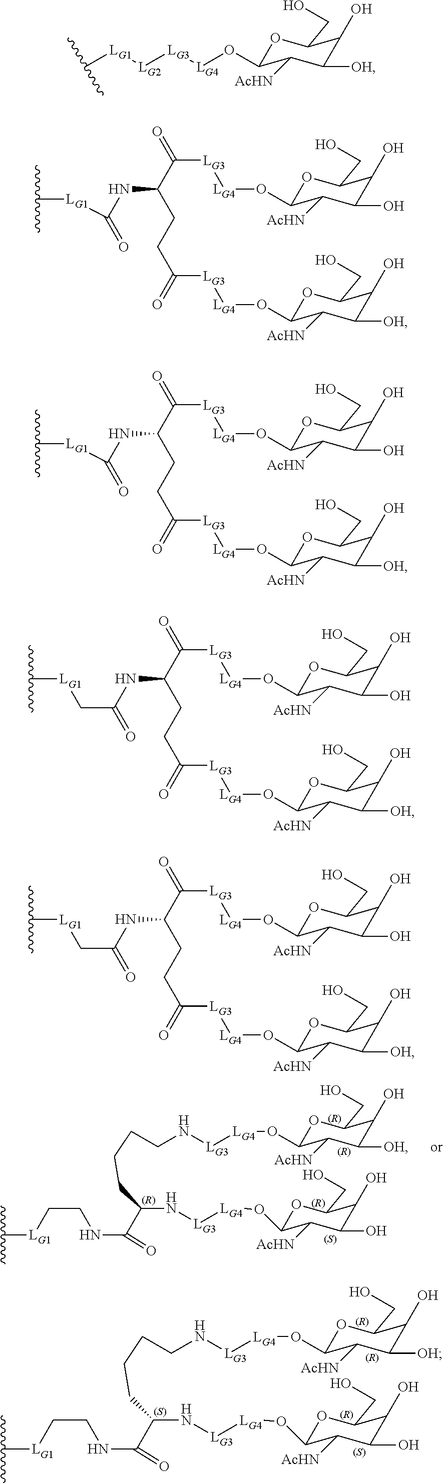

wherein L_{G1} is a bond, —CH₂—, —CH₂CH₂—, —C(O)—, —CH₂O—, —CH₂O—CH₂CH₂O—, or —NHC(O)—(CH₂NHC(O))ₐ—;
L_{G2} is a bond or —CH₂CH₂C(O)—;
L_{G3} is a bond, —(NHCH₂CH₂)A—, —(NHCH₂CH₂CH₂)_{b}—, or —C(O)CH₂—;
L_{G4} is —(OCH₂CH₂)_{c}—, —(OCH₂CH₂CH₂)_{c}—, —(OCH₂CH₂CH₂CH₂)_{c}—, —(OCH₂CH₂CH₂CH₂CH₂)_{c}—, or —NHC(O)—(CH₂)_{d}—;
   wherein a=0, 1, 2, or 3;
   b=1, 2, 3, 4 or 5;
   c=1,2,3,4 or 5;
   d=1, 2,3,4,5,6,7, or 8:
A is a bond, —CH₂O—, or —NHC(O)—:
A′ is a bond, —C(O)NH—, —NHC(O)—, or —O(CH₂CH₂O)_{c}—;
   wherein e is 1, 2, 3, 4, or 5;
B is a bond, —CH₂—, —C(O)—, —M—, —CH₂-M—, or —C(O)—M—;
   wherein M is

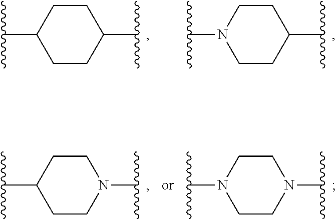 [SMILES: *C1CCC(*)CC1 ; *C1CCN(*)CC1 ; *C1CCN(*)CC1 ; *N1CCN(*)CC1]

R_{G1} and R_{G2} together form —CH₂CH₂O— or —CH₂CH(RG)—O—, and R_{G3} is H.
or R_{G1} and R_{G3} together form —C₁₋₂ alkylenc-, and R_{G2} is H;
wherein R_{G} is —OR_{G}′, —CH₂OR_{G}′, or —CH₂CH₂OR_{G}′; wherein R_{G}′ is H, a hydroxy-protecting group, or a solid support, and the hydroxy-protecting group is preferably —C(O)CH₂CH₂C(O)OH or 4,4′-dimethoxytrityl;
m1=0, 1, 2, 3, 4, 5, 6, 7, 8, 9 or 10:
n1=0, 1, 2, 3, 4, 5, 6, 7, 8, 9 or 10.

In another embodiment. GaINAc is a conjugation group of formula (I′):

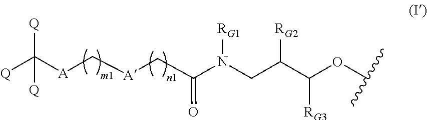

wherein,

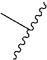

represents the position of attachment to biomolecules; Q is independently H,

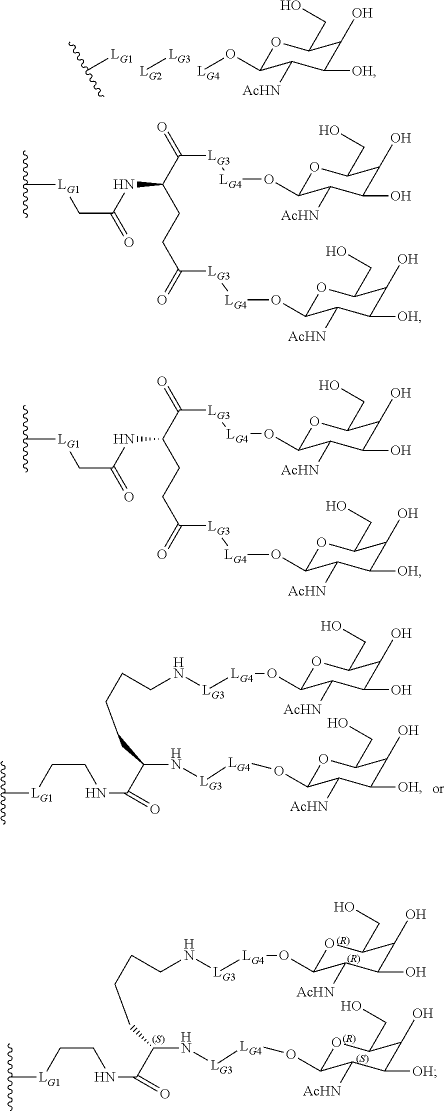

wherein L_{G1} is a bond, —CH₂—, —CH₂CH₂—, —C(O)—, —CH₂O—, —CH₂O—CH₂CH₂O—, or —NHC(O)—(CH₂NHC(O))ₐ—;
L_{G2} is a bond or —CH₂CH₂C(O)—;
L_{G3} is a bond, —(NHCH₂CH₂)_{b}—, —(NHCH₂CH₂CH₂)_{b}—, or —C(O)CH₂—;
L_{G4} is —(OCH₂CH₂)—, —(OCH₂CH₂CH₂)_{c}—, —(OCH₂CH₂CH₂CH₂)_{c}—, —(OCH₂CH₂CH₂CH₂CH₂)_{c}—, or —NHC(O)—(CH₂)_{d}—;
   wherein a=0, 1, 2, or 3;
   b=1, 2,3,4 or 5;
   c=1, 2, 3,4 or 5:
   d=1, 2, 3, 4, 5, 6, 7, or 8;
A is —CH₂O—or —NHC(O)—;
A′ is a bond, —C(O)NH— or —NHC(O)—;
R_{G1} and R_{G2} together form —CH₂CH₂O—or —CH₂CH(RG)—O—, and R_{G3} is H;
or R_{G1} and R_{G3} together form —C₁₋₂ alkylene-, and R_{G2} is H;
wherein R_{G} is —OR_{G}′, —CH₂OR_{G}′, or —CH₂CH₂OR_{G}′; wherein R_{G}′ is H, a hydroxy-protecting group, or a solid support, and the hydroxy-protecting group is preferably —C(O)CH₂CH₂C(O)OH or 4,4′-dimethoxytrityl;
m1=0, 1, 2, 3, 4, 5, 6, 7, 8, 9 or 10;
n1=0, 1,2,3,4, 5,6,7,8,9or 10.

In another embodiment. GaINAc is a conjugation group of formula (X), wherein Q is independently H,

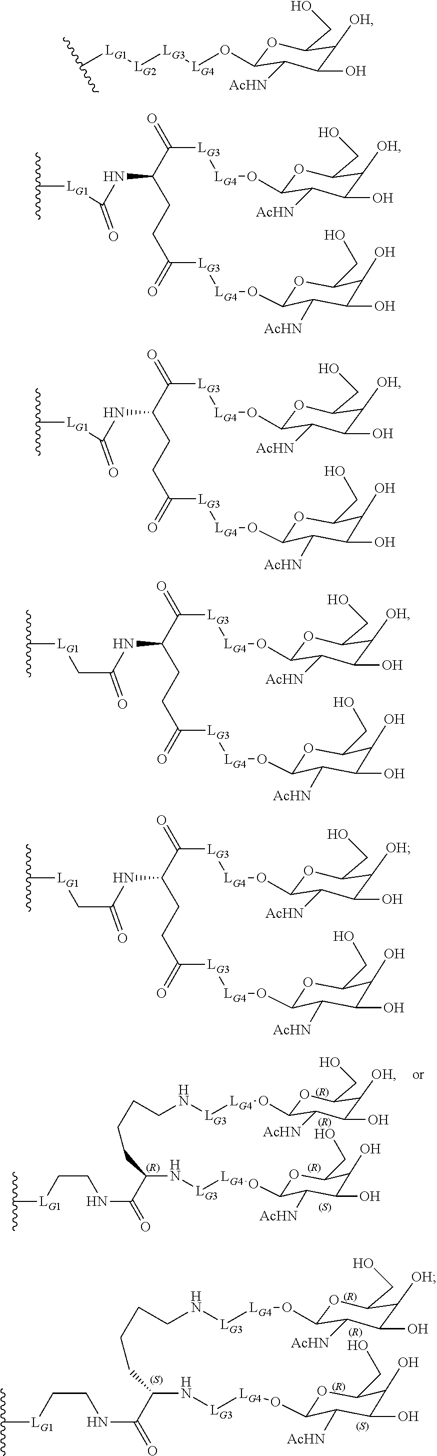

wherein L_{G1} is a bond, —CH₂—, —CH₂CH₂—, —C(O)—, —CH₂O—, —CH₂O—CH₂CH₂O—, or —NHC(O)—(CH₂NHC(O))ₐ—;
L_{G2} is a bond or —CH₂CH₂C(O)—;
L_{G3} is a bond, —(NHCH₂CH₂)_{b},—, —(NHCH₂CH₂CH₂)_{b}—, or —C(O)CH₂—:
L_{G4} is —(OCH₂CH₂)_{c}—, —(OCH₂CH₂CH₂)_{c}—, —(OCH₂CH₂CH₂CH₂)_{c}—, —(OCH₂CH₂CH₂CH₂CH₂)_{c}—, or —NHC(O)—(CH₂)_{d}—;
   wherein a=0, 1, 2, or 3;
   b=1, 2, 3,4 or 5;
   c=1.2, 3,4or 5;
   d=1, 2,3,4,5,6,7, or 8:
A is a bond, —CH₂O—, or —NHC(O)—;
A′ is a bond, —C(O)NH—, —NHC(O)—, or —O(CH₂CH₂O)_{c}—;
   wherein e is 1, 2, 3, 4, or 5;
B is a bond, —CH₂—, —M—, —CH₂-M- or —C(O)—M—;
wherein M is

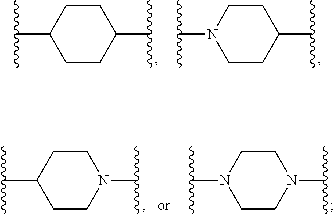

R_{G1} and R_{G2} together form —CH₂CH₂O—or —CH₂CH(RG)—O—, and R_{G3} is H;
or R_{G1} and R_{G3} together form —C₁₋₂ alkylene-, and R_{G2} is H;
wherein R_{G} is —OR_{G}′, —CH₂OR_{G}′, or —CH₂CH₂OR_{G}′; wherein R_{G}′ is H, a hydroxy-protecting group, or a solid support, and the hydroxy-protecting group is preferably —C(O)CH₂CH₂C(O)OH or 4,4′-dimethoxytrityl;
m1=0, 1, 2, 3, 4, 5, 6, 7, 8,9 or 10;
n1=0, 1,2,3,4, 5,6,7,8,9or 10.

In another embodiment. GaINAc is a conjugation group of formula (X), wherein:
Q is independently H,

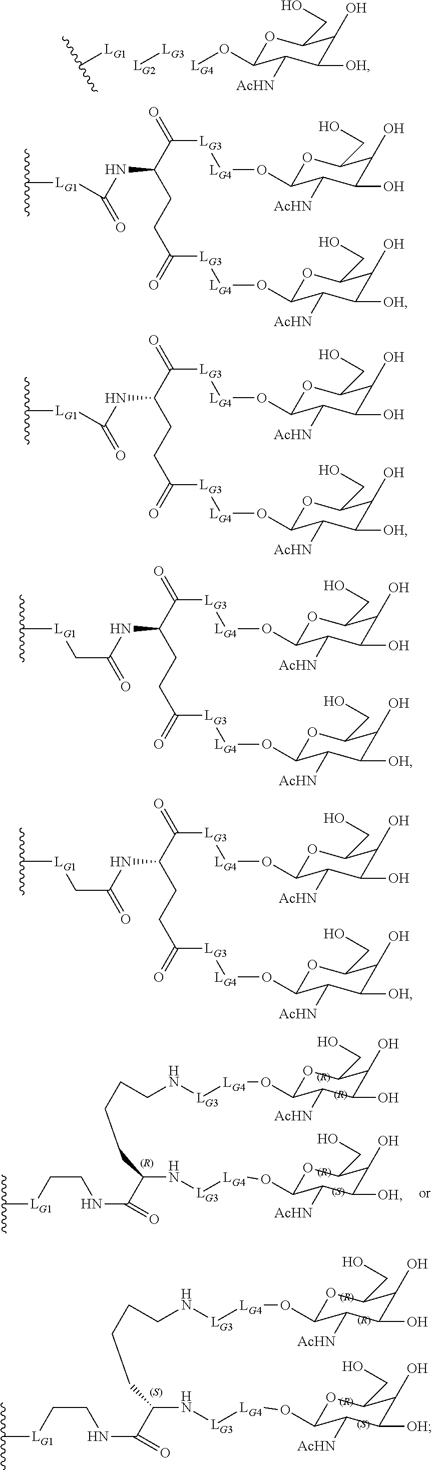

wherein L_{G1} is a bond, —CH₂—, —CH₂CH₂—, —C(O)—, —CH₂O—, —CH₂O—CH₂CH₂O—, or —NHC(O)—(CH₂NHC(O))ₐ—;
L_{G2} is a bond or —CH₂CH₂C(O)—;
L_{G3} is a bond, —(NHCH₂CH₂)_{b}—, —(NHCH₂CH₂CH₂)_{b},—, or —C(O)CH₂—; L_{G4} is —(OCH₂CH₂)_{c}—, —(OCH₂CH₂CH₂)_{c}—, —(OCH₂CH₂CH₂CH₂)_{c}—, —(OCH₂CH₂CH₂CH₂CH₂)_{c}—, or —NHC(O)—(CH₂)_{d}—;
   wherein a=0, 1, 2, or 3;
   b=1, 2, 3, 4 or 5;
   c=1,2,3,4 or 5;
   d=1, 2,3,4,5,6,7, or 8;
A is a bond, —CH₂O—, or —NHC(O)—;
A′ is —O(CH₂CH₂O)_{c}—;
   wherein e is 1, 2, 3, 4, or 5;
B is a bond, —CH₂—, —C(O)—, —M——CH₂—M—, or —C(O)—M—;
wherein M is

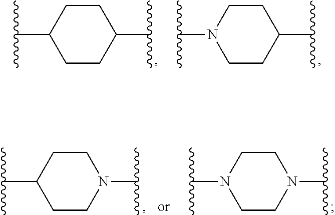

R_{G1} and R_{G2} together form —CH₂CH₂O—or —CH₂CH(RG)—O—, and R_{G3} is H;
or R_{G1} and R_{G3} together form —C₁₋₂ alkylenc-, and R_{G2} is H;
   wherein R_{G} is —OR_{G}′, —CH₂OR_{G}′, or —CH₂CH₂OR_{G}′; wherein R_{G}′ is H, a hydroxy-protecting group, or a solid support, and the hydroxy-protecting group is preferably —C(O)CH₂CH₂C(O)OH or 4,4′-dimethoxytrityl;
m1=0, 1,2,3,4,5,6,7, 8,9 or 10;
n1=0, 1,2,3,4, 5,6,7,8,9or 10.

Any technical solution or any combination thereof in any of the above specific embodiments can be combined with any technical solution or any combination thereof in other specific embodiments. For example, anv technical solution or any combination thereof of X can be combined with any technical solution or any combination thereof of Q₁, Q₂, Y₁, Y₂, L₁, L₂, R₁, R₂, R₃, R₄, R₅, Base, and Base′. The present invention is intended to include all combinations of these technical solutions, which are not listed one by one due to space limitations.

The present invention also provides a vector comprising a nucleotide sequence encoding the siRNA of the invention. The vector of the present invention can amplify or express a nucleotide encoding the siRNA of the invention linked thereto.

For example, a siRNA targeting the PCSK9 gene can be expressed from a transcription unit inserted into a DNA or RNA vector. Expression can be transient (within hours to weeks) or sustained (weeks to months or longer), depending on the particular construct used and the target tissue or cell type. A nucleotide encoding the siRNA can be introduced into a linear construct, a circular plasmid, or a viral vector. A nucleotide encoding the siRNA can be stably expressed by integration into the cell genome, or can be stably inherited and expressed extrachromosomally. Generally speaking, a vector expressing the siRNA is usually a DNA plasmid or a viral vector.

Viral vector systems comprising a sequence encoding the siRNA include, but are not limited to: (a) adenoviral vectors; (b) retroviral vectors; (c) adeno-associated viral vectors; (d)herpes simplex virus vectors; (e) SV40 vectors; (f) polyomavirus vectors; (g) papillomavirus vectors;(h) picornavirus vectors (i) poxvirus vectors; and (j)helper virus-dependent or gutless adenoviral vectors.

The present invention also provides a cell comprising the siRNA or vector of the invention, wherein the siRNA or vector of the invention can be transcribed in the cell.

The present invention specifically relates to the following technical solutions:

1. A nucleotide dimer of formula (A):

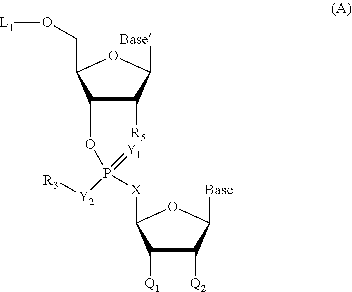

wherein one of Q₁ and Q₂ is R₄, and the other is O-L₂;
L₁ is H or P₁, or a bond to the phosphorus atom P of the phosphate group at the 2′ or 3′ end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂;
X is selected from —(CR₁R₂)ₘ—or —CR₁=CR₂—;
Y₁ is O, S, or NR;
Y₂ is O, S, or a bond;
R₁ and R₂ are independently selected from H, D, halogen, OH, CN, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl, which are optionally substituted with 1, 2, 3, 4, 5, 6, 7, 8, or more R′;
R₃ is selected from H, C₁₋₆ alkyl, C₁₋₆ haloalkyl, C₂₋₆ alkenyl, C₂₋₆ alkynyl, C₃₋₁₀ cycloalkyl, 3-membered to 10-membemd heterocyclyl, C₆₋₁₀ aryl, or 5-membercd to 14-membered heteroaryl, which are optionally substituted with 1, 2, 3, 4, 5, 6, 7, 8, or more R′:
R₄ and R₅ are independently selected from H, D, OH, halogen, C₁₋₆ alkyl, C₁₋₆ haloalkyl, or C₁₋₆ alkoxyl, preferably H, F, or OMe.
P₁ is a hydroxy-protecting group, preferably DMTr;
P₂ is a reactive phosphorus group, preferably —P(OCH;CH₂CN)(N(iPr)₂);
Base and Base′ are independently selected from H, a modified or unmodified base or leaving group, preferably modified or unmodified A. U, T, G, and C:
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, 5-membered to 14-membered heteroaryl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —S(O)ₙR^{a}, —S(O)ₙOR^{a}, —S(O)ₙNR^{a}R^{b}, —O—S(O)ₙR^{b}, —NR^{a}R^{b}, —NR^{a}C(O)R^{b}, —NR^{a}—C(O)OR^{b}, —NR^{a}S(O)ₙR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl: or R^{a} and R^{b} and the nitrogen atom to which they are attached form a 3-membered to 10-membered heterocyclyl;
m is selected from 1, 2, 3, 4 or 5;
n is independently selected from 1 or 2.
2. A nucleotide dimer according to technical solution 1, wherein the nucleotide dimer has a structure of formula (I) or (II):

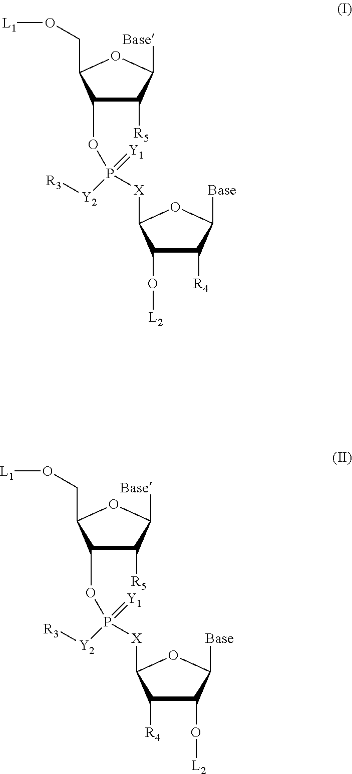

wherein each group is as defined in technical solution 1.
3. A nucleotide dimer according to technical solution 2, wherein,
X is selected from —(CR₁R₂)ₘ—or —CR₁=CR₂—; R₁ and R₂ are independently selected from H, D, halogen, OH, CN, C₁₋₆ alkyl, C₁₋₆ haloalkyl, C₂₋₆ alkenyl, or C₂₋₆ alkynyl, which are optionally substituted with 1, 2, 3, 4, 5, or more R′;
R′ is selected from D, halogen, CN, C₁₋₆ alkyl, C₁₋₆ haloalkyl, C₂₋₆ alkenyl, C₂₋₆ alkynyl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —NR^{a}R^{b}, —NR^{a}C(O)R^{b}, —NR^{a}—C(O)OR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, or C₂₋₆ alkynyl;
m is selected from 1, 2, 3, 4 or 5;
preferably,
X is selected from —(CR₁R₂)ₘ—or —CR₁═CR₂—;
R₁ and R₂ are independently selected from H, D, halogen, OH, CN, C₁₋₆ alkyl, or C₁₋₆ haloalkyl, which are optionally substituted with 1, 2, 3, or more R′;
R′ is selected from D, halogen, CN, C₁₋₆ alkyl, C₁₋₆ haloalkyl, C₂₋₆ alkenyl, C₂₋₆ alkynyl, —OR^{a}, or —NR^{a}R^{b};
R^{a} and R^{b} are independently selected from H, C₁₋₆ alkyl, or C₁₋₆ haloalkyl;
m is selected from 1, 2, or 3;
more preferably.
X is selected from —CH₂—. —CH(OH)—, —CH₂—CH₂—, or —CH═CH—.
4. A nucleotide dimer according to technical solution 2 or 3, wherein,
Y₁ is O or NR;
Y₂ is O, S, or a bond:
R is selected from H, C₁₋₆ alkyl, or C₁₋₆ haloalkyl;
preferably,
Y₁ is O or NR;
Y₂ is O, S, or a bond;
R is selected from H or C₁₋₆ alkyl;
more preferably,
Y₁ is O;
Y₂ is O.
5. A nucleotide dimer according to any one of technical solutions 2 to 4, wherein,
R₃ is selected from H, C₁₋₆ alkyl, C₁₋₆ haloalkyl, C₂₋₆ alkenyl, or C₂₋₆ alkynyl, which are optionally substituted with 1, 2, 3, 4, 5, or more R;
preferably,
R₃ is selected from H, C₁₋₆ alkyl, or C₁₋₆ haloalkyl, which is optionally substituted with 1, 2, 3, or more R′; more preferably,
R₃ is C₁₋₄ alkyl, preferably Me.
6. A nucleotide dimer according to any one of technical solutions 2 to 5, wherein,
R₄ and R₅ are independently selected from H, D, OH, halogen, C₁₋₆ alkyl, C₁₋₆ haloalkyl, or C₁₋₆ alkoxyl;
preferably,
R₄ and R₅ are independently selected from H, OH, halogen, C₁₋₆ alkyl, C₁₋₆ haloalkyl, or C₁₋₆ alkoxyl, preferably H, F, OH, or OMe;
more preferably,
R₄ is selected from H, OH, or C₁₋₄ alkoxyl, preferably H or OMe:
R₅ is selected from halogen, OH, or C₁₋₄ alkoxyl, preferably F or OMe. 7. A nucleotide dimer according to any one of technical solutions 2 to 6, wherein.
Base and Base′ are independently selected from

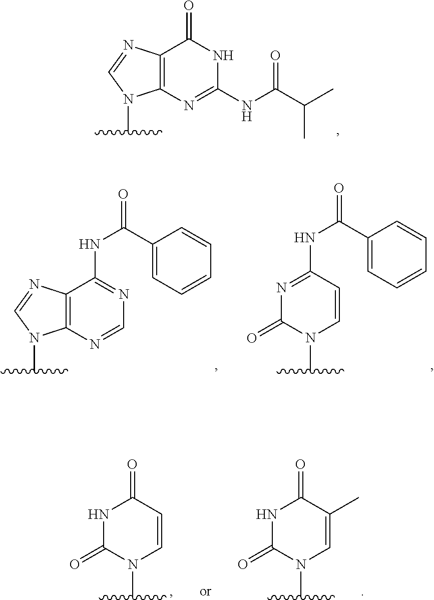

8. A nucleotide dimer according to any one of technical solutions 2 to 7, wherein, L₁ is H or P₁, or a bond to the phosphorus atom P of the phosphate group at the 2 or 3 end of the ribose of another nucleotide or oligonucleotide, preferably P₁: L₂ is H or P₂, or a bond to the phosphorus atom P of the phosphate group at the 5′ end of the ribose of another nucleotide or oligonucleotide, preferably P₂:
X is selected from —(CR₁R₂)ₘ—or —CR₁=CR₂—;
Y₁ is O, S, or NR:
Y₁ is O, S. or a bond;
R₁ and R₂ are independently selected from H, D, halogen, OH, CN, C₁₋₆ alkyl, C₁₋₆ haloalkyl, C₂₋₆ alkenyl, or C₂₋₆ alkynyl, which are optionally substituted with 1, 2, 3, 4, 5, or more R′;
R₃ is selected from H, C₁₋₆ alkyl, C₁₋₆ haloalkyl, C₂₋₆ alkenyl, or C₂₋₆ alkynyl, which are optionally substituted with 1, 2, 3, 4, 5, or more R′;
R₄ and R₅ are independently selected from H, D, OH, halogen, C₁₋₆ alkyl, C₁₋₆ haloalkyl, or C₁₋₆ alkoxyl, preferably H, F, or OMe;
P₁ is a hydroxy-protecting group, preferably DMTr;
P₂ is a reactive phosphorus group, preferably —P(OCH₂CH₂CN)(N(iPr)₂);
Base and Base′ are independently selected from H, a modified or unmodified base or leaving group, preferably modified or unmodified A, U, T, G, and C;
R is selected from H, C₁₋₆ alkvl, or C₁.e haloalkvl:
R′ is selected from D, halogen, CN. C₁₋₆ alkyl, C₁₋₆ haloalkyl, C₂₋₆ alkenyl, Cz₂₋₆ alkynyl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —NR^{a}R^{b}, —NR^{a}C(O)R^{b}, —NR^{a}—C(O)OR^{b}, or —NR^{a}C(O)NR^{a}R^{b}:
R^{a} and R^{b} are independently selected from H, C₁₋₆ alkyl, C₁₋₆ haloalkyl, C₂₋₆ alkenyl, or C₂₋₆ alkynyl;
m is selected from 1, 2, 3, 4, or 5.
9. A nucleotide dimer according to any one of technical solutions 2 to 8, wherein,
L₁ is H or P₁, or a bond to the phosphorus atom P of the phosphate group at the 2 or 3 end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂:
X is selected from —(CR₁R₂)ₘ—or-CR₁═CR₂—;
Y₁ is O or NR;
Y₂ is O, S. or a bond;
R₁ and R₂ are independently selected from H, D, halogen, OH, CN, C₁₋₆ alkyl, or C₁₋₆ haloalkyl, which are optionally substituted with 1, 2, 3, or more R′;
R₃ is selected from H, C₁₋₆ alkyl, or C₁₋₆ haloalkyl, which is optionally substituted with 1, 2, 3, or more R′;
R₄ and R₅ are independently selected from H, D, halogen, C₁₋₆ alkyl, C₁₋₆, haloalkyl, or C₁₋₆ alkoxyl, preferably H, F, or OMe:
P₁ is a hydroxy-protecting group, preferably DMTr:
P₂ is a reactive phosphorus group, preferably —P(OCH₂CH₂CN)(N(iPr)₂);
Base and Base′ are independently selected from H, a modified or unmodified base or leaving group, preferably modified or unmodified A, U. T, G, and C;
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, —OR^{a}, or —NR^{a}R^{b};
R^{a} and R^{b} are independently selected from H, C₁₋₆ alkyl, or C₁₋₆ haloalkyl;
m is selected from 1, 2, or 3.
10. A nucleotide dimer according to any one of technical solutions 2 to 9, wherein,
L₁ is H or P₁, or a bond to the phosphorus atom P of the phosphate group at the 2 or 3end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂;
X is selected from —(CR₁R₂)ₘ—or —CR₁═CR₂—, preferably —CH₂—, —CH(OH)—, —CH₂—CH₂—, or —CH═CH—;
Y₁ is O;
Y₂ is O;
R₁ and R₂ are independently selected from H, D, halogen, OH, CN, C₁₋₄ alkyl, or C₁₋₄ haloalkyl;
R₃ is C₁₋₄ alkyl, preferably Me;
R₄ is selected from H, OH, or C₁₋₄ alkoxyl, preferably H or OMe;
R₅ is selected from halogen, OH, or C₁₋₄ alkoxyl, preferably F or OMe;
P₁ is a hydroxy-protecting group, preferably DMTr;
P₂ is a reactive phosphorus group, preferably —P(OCH₂CH₂CN)(N(iPr)₂);

Base and Base are independently selected from

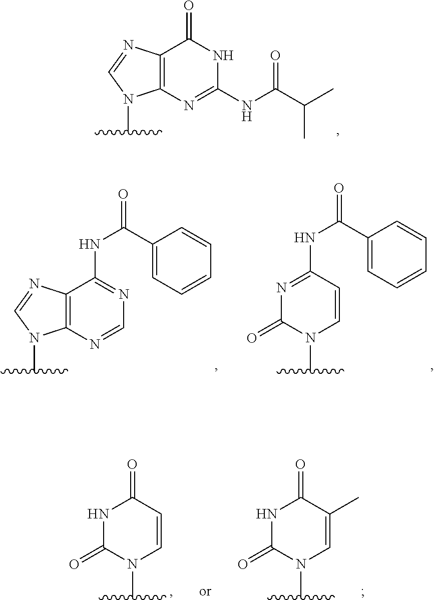

m is selected from 1, 2, or 3.
11. A nucleotide dimer according to any one of technical solutions 1 to 10, wherein the nucleotide dimer is selected from:

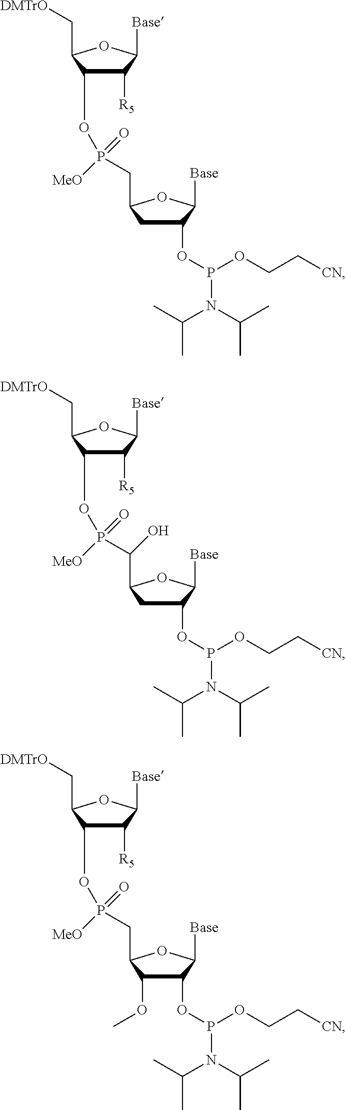

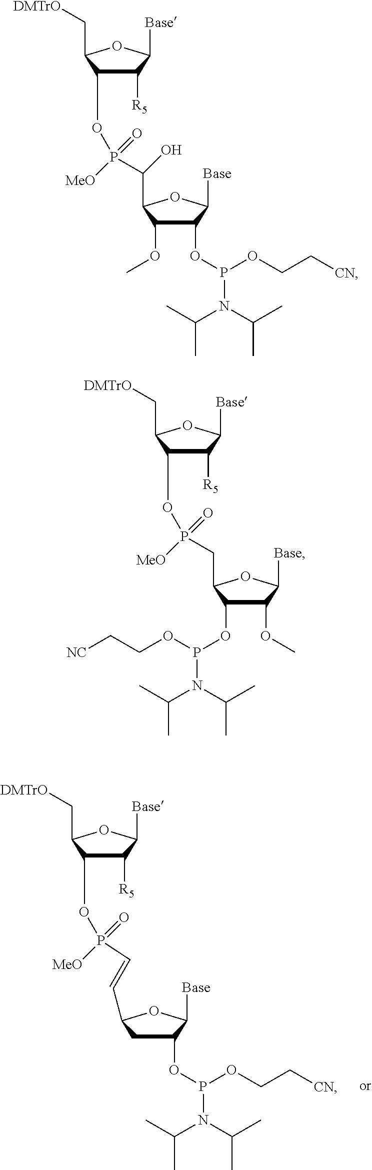

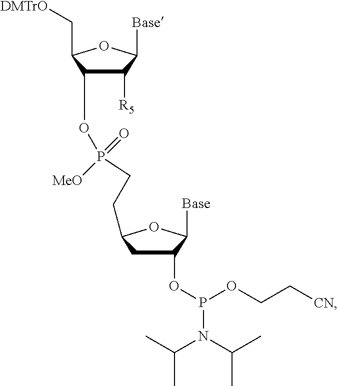

wherein Base and Base′ are as defined in any one of technical solutions 1 to 10, preferably

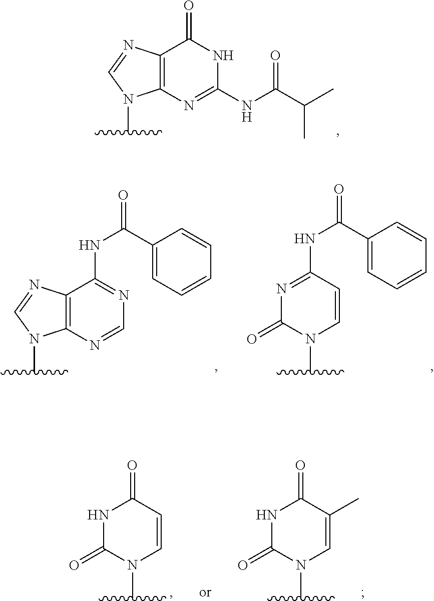

R₅ is as defined in any one of technical solutions 1 to 10, preferably F or OMe.
12. A dsRNA molecule, or a pharmaceutically acceptable salt, tautomer, or stereoisomer thereof, comprising a sense strand and an antisense strand, each strand having 14 to 30 nucleotides; wherein the antisense strand comprises one or more nucleotide monomers of formula (III) or (IV):

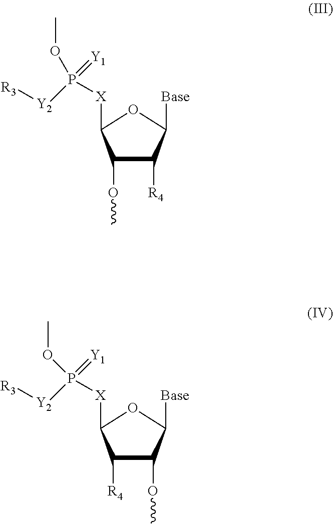

wherein the nucleotide monomers are connected from ----- to [symbol] in the 5′ to 3′ direction;
each group is as defined in any one of technical solutions 1 to 11;
preferably, the nucleotide monomers are selected from:

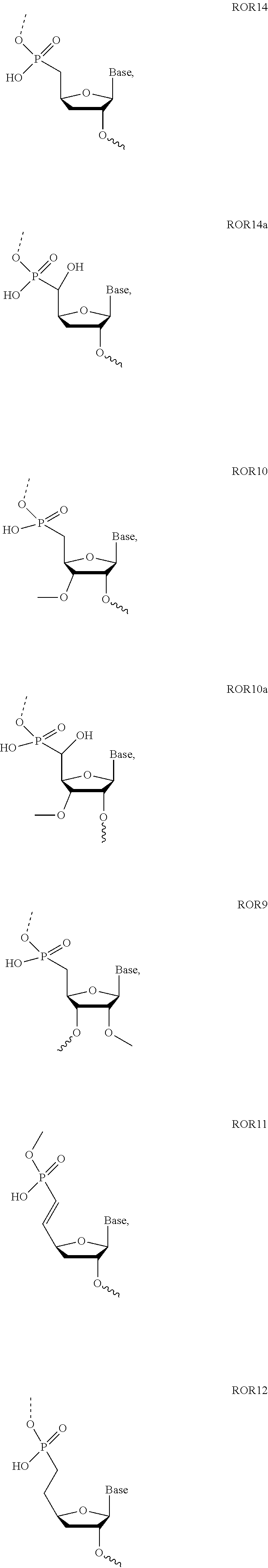

wherein Base is selected from

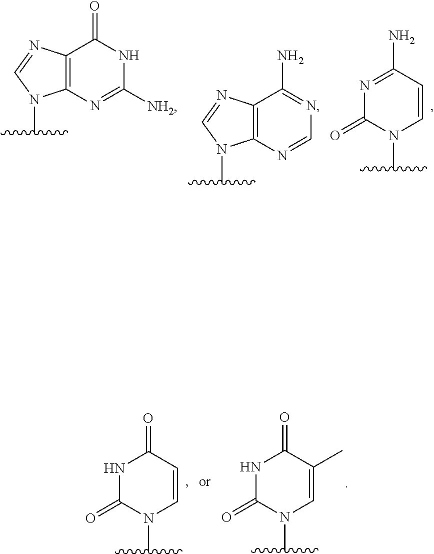

13. A dsRNA molecule according to technical solution 12, wherein the sense strand and the antisense strand each have 20 to 25 nucleotides.
14. A dsRNA molecule according to technical solution 12 or 13, wherein the nucleotide monomer is located at positions 2 to 8, preferably position 6 or 7, more preferably position 7, of the 5′ end of the antisense strand.
15. A dsRNA molecule according to any one of technical solutions 12 to 14, wherein the dsRNA exhibits enhanced stability compared with a dsRNA having the same sequence but not comprising the nucleotide monomer of technical solution 12.
16. A dsRNA molecule according to any one of technical solutions 12 to 15, wherein the dsRNA has a melting temperature of about 40° C. to about 80° C. preferably about 55° C. to 67° C.
17. A dsRNA molecule according to any one of technical solutions 12 to 16, wherein the dsRNA exhibits reduced off-target toxicity compared with a dsRNA having the same sequence but not comprising the nucleotide monomer of technical solution 12.
18. A dsRNA molecule according to any one of technical solutions 12 to 17, wherein the dsRNA exhibits enhanced effectiveness compared with a dsRNA having the same sequence but not comprising the nucleotide monomer of technical solution 12.
19. A dsRNA molecule according to any one of technical solutions 12 to 18, wherein the antisense strand has a sequence sufficiently complementary to the sense strand and target mRNA and is capable of inducing degradation of the target mRNA.
20. A dsRNA molecule according to technical solutions 12 to 19, wherein the target mRNA is encoded by an endogenous gene or by a pathogen gene.
21. A dsRNA molecule according to any one of technical solutions 12 to 20, wherein the sense strand and/or antisense strand comprises a 3′ and/or 5′ over-hang.
22. A dsRNA molecule according to any one of technical solutions 12 to 21, wherein the dsRNA is further conjugated to a ligand; preferably, the ligand comprises one or more GalNAc.
23. A nucleic acid molecule comprising in its nucleotide sequence one or more nucleotide monomers of technical solution 12.
24. A nucleic acid molecule according to technical solution 23, wherein the nucleic acid is selected from DNA, RNA, and a DNA/RNA hybrid.
25. A nucleic acid molecule according to technical solution 24, wherein the nucleic acid molecule is single- or double-stranded.
26. A nucleic acid molecule according to any one of technical solutions 23 to 25, wherein the nucleic acid molecule is selected from small interfering RNA (siRNA) and short hairpin RNA (shRNA).
27. A vector comprising a nucleotide sequence encoding a dsRNA of any one of technical solutions 12 to 22.
28. A cell comprising a dsRNA of any one of technical solutions 12 to 22 or a vector of technical solution 27.
29. A pharmaceutical composition comprising the dsRNA molecule according to any one of technical solutions 12 to 22 and a pharmaceutically acceptable carrier or excipient.
30. A kit comprising the dsRNA molecule according to any one of technical solutions 12 to 22.
31. A method for inhibiting the expression of a target gene in a cell, comprising a step of introducing the dsRNA molecule according to any one of technical solutions 12 to 22 into the cell.
32. A method for inhibiting the expression of a target gene in a cell, comprising expressing the dsRNA molecule according to any one of technical solutions 12 to 22 in the cell.
33. A method for reducing off-target toxicity in a cell, comprising a step of introducing the dsRNA molecule according to any one of technical solutions 12 to 22 into the cell.
34. A method for reducing off-target toxicity in a cell, comprising expressing the dsRNA molecule according to any one of technical solutions 12 to 22 in the cell.

### Examples

The following examples are intended to illustrate the invention and do not limit the scope of the invention.

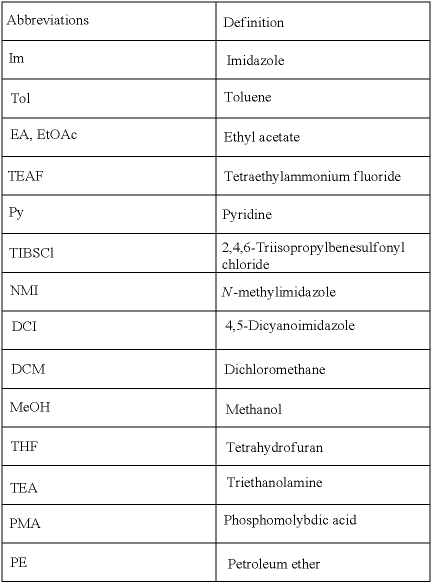

### EXAMPLE 1 Preparation of Compound E1

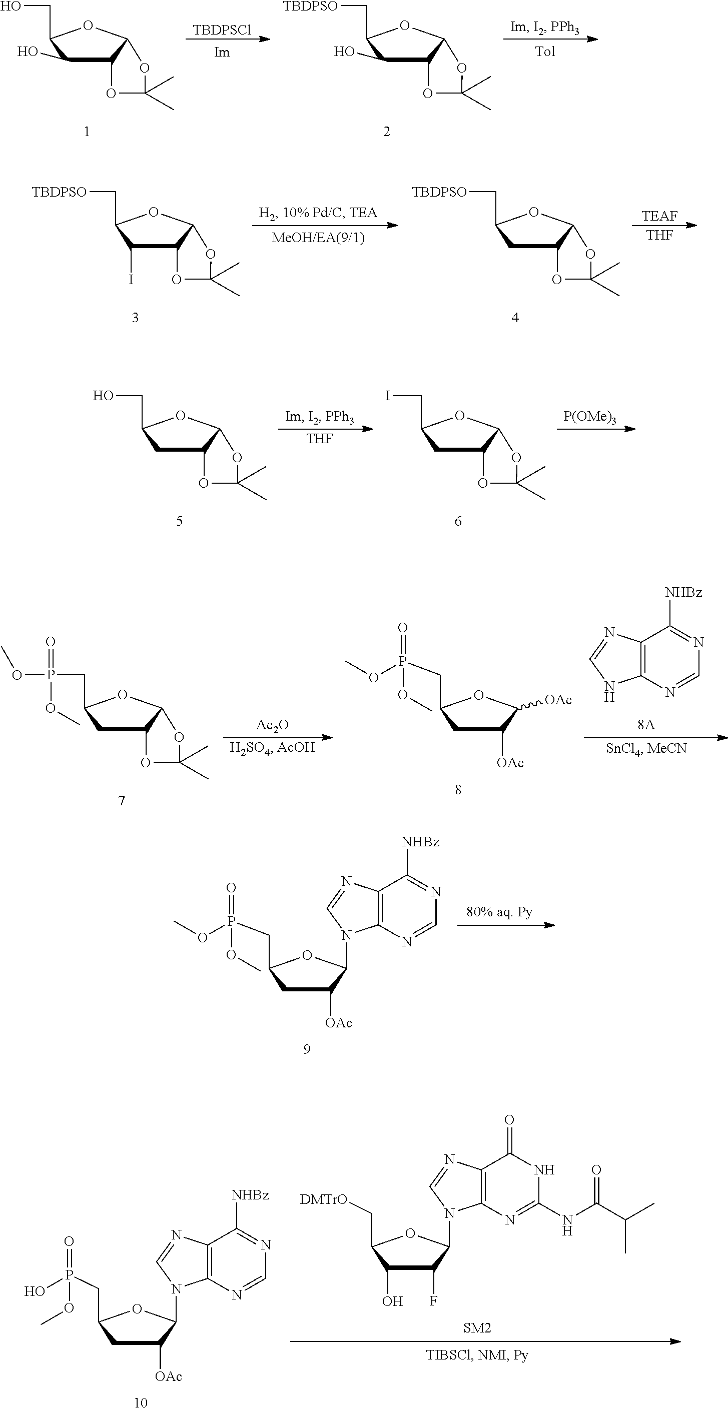

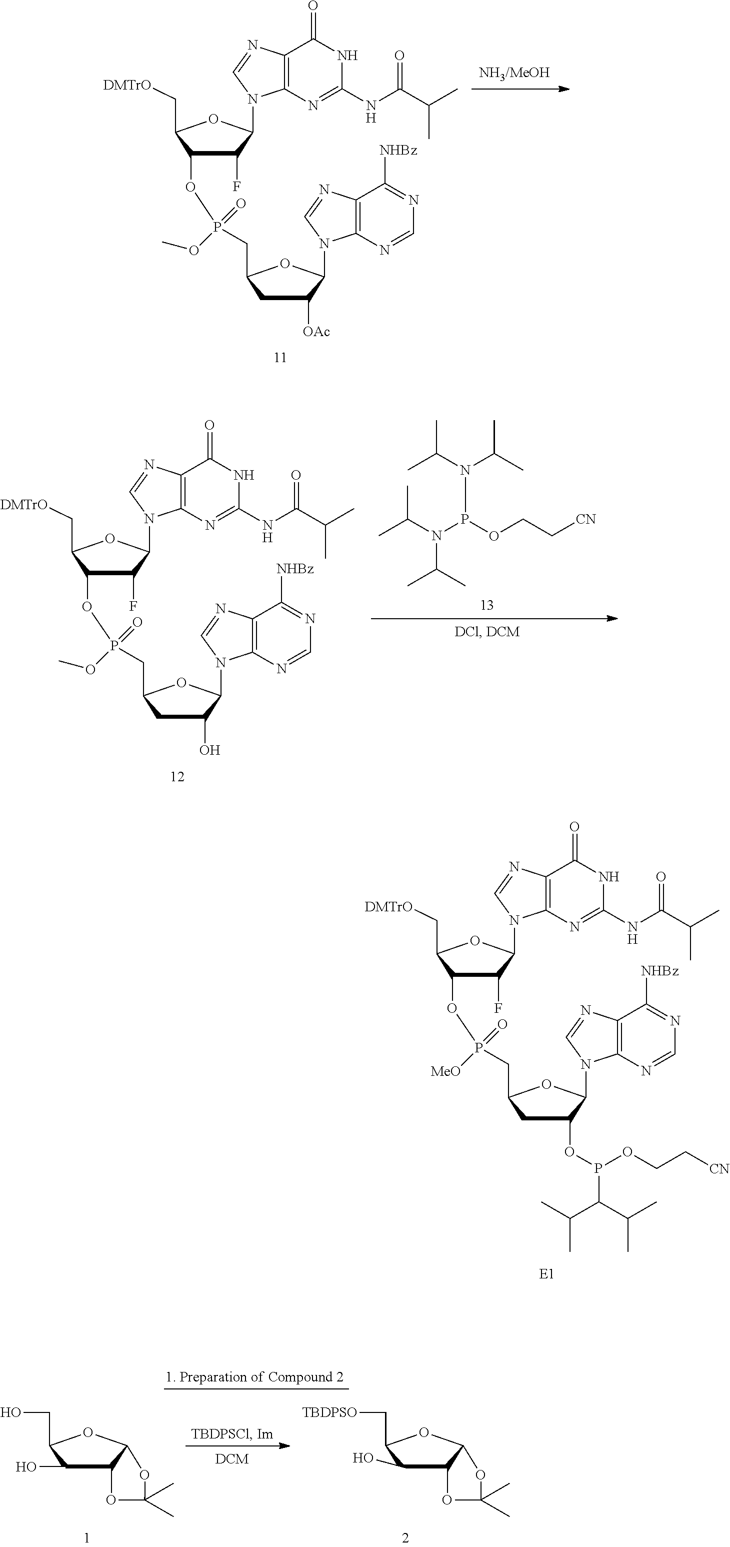

Compound 1 (50.0 g, 263 mmol) was dissolved in DCM (800 mL) at 25° C. Imidazolc (26.9 g, 394 mmol) IPGP-8,C₃ and TBDPSCI (75.2 mL, 289 mmol) were added in sequence. The reaction mixture was stirred at 25° C. for 18 h until TLC (DCM/MeOH=10/1, PE/EA=3/l) showed complete consumption of reactants and formation of new spots. The reaction mixture was spin-dried to obtain a crude product. The crude product was purified by MPLC (PE/EA=I/O−5/1) to obtain compound 2 (95.0 g, yield 84.31%) as a colorless oily liquid.

¹H NMR (400 MHz, CDCb.) δ7.70 (dd, J=7.6, 1.6 Hz, 4H), 7.35-7.46 (m, 6H), 5.86 (d, J=3.6 Hz, 1 H), 4.61 (dd, J=4.8.4.0 Hz, 1H), 4.15 (td.J=8.8, 5.2 Hz, 1H), 3.93-4.01 (m, 1H), 3.81-3.92 (m, 2H), 1.60 (s, 3H), 1.39 (s, 3H), 1.06 (s, 9H).

2. Preparation of Compound 3

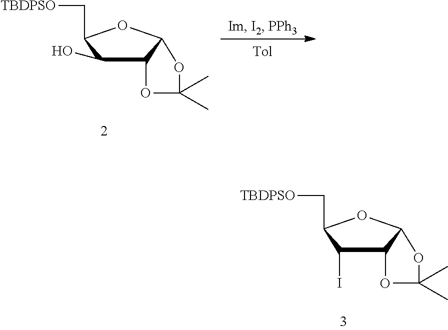

Compound 2 (95.0 g, 222 mmol) was dissolved in toluene (1.50 L) at 25° C. Imidazole (30.2 g, 443 mmol), triphenylphosphine (116 g, 443 mmol), and elemental iodine (84.4 g, 322 mmol) were added in sequence. The reaction mixture was stirred at 100° C. for 18 h until TLC (PE/EA=5/1) showed complete consumption of reactants and formation of new spots. To the reaction mixture, 20.0 mL of saturated NaHSO3 solution and 500 mL of water were added until the reaction mixture was separated into layers. The organic phase was washed with saturated NaCl solution (50.0 mL×3), dried over anhydrous Na₂SO₄, and then spin-dried to obtain a crude product. The crude product was purified by MPLC (PE/EA=I/O-10/1) to obtain compound 3 (115 g, yield 96.35%) as a colorless oily liquid.

¹H NMR (400 MHz, CD₃OD) δ7.64-7.72 (m, 4H), 7.37-7.49 (m, 6H), 5.95 (d, J=3.6 Hz, I H), 5.06 (d, J=3.6 Hz, 1H), 4.42 (d, J=3.2 Hz, 1H), 3.87 (dd. J=10.4, 5.6 Hz, 1H), 3.66 (dd, J=10.4, 6.4 Hz, 1H), 3.53 (td, J=6.0, 3.2 Hz, 1H), 1.44 (s, 3H), 1.29 (s, 3H), 1.02-1.07 (m, 9H).

3. Preparation of Compound 4

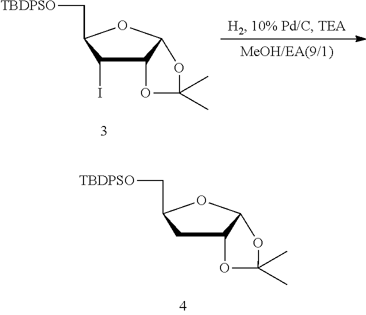

Compound 3 (12.5 g, 23.2 mmol) was dissolved in a mixed solvent of MeOH (225 mL) and EtOAc (25 mL) at 25° C. TEA (6.45 mL, 46.4 mmol) and Pd/C 10% (2.00 g, 18.8 mmol) were added, and the reaction mixture was stirred at 25° C. under a hydrogen (14.696 psi) atmosphere for 14 h until TLC (PE/EA=10/1) showed complete consumption of reactants and formation of new spots. The reaction mixture was filtered, and the filtrate was spin-dried to obtain a crude product. The crude product was purified by MPLC (PE/EA=I/O-10/1) to obtain compound 4 (8.75 g, yield 83.06%) as a colorless oily liquid.

¹H NMR (400 MHz, CD₃OD) δ7.64-7.72 (m. 4 H), 7.36-7.47 (m, 6H), 5.80 (d, J=3.6 Hz, I H), 4.77 (t. J=4.4 Hz, 1H), 4.25-4.32 (m, 1H), 3.68-3.82 (m, 2H), 2.00 (dd, J=13.6, 4.8 Hz, I H), 1.78-1.87 (m, 1H), 1.45 (s, 3H), 1.30 (s, 3H), 1.04 (s, 9H).

4. Preparation of Compound 5

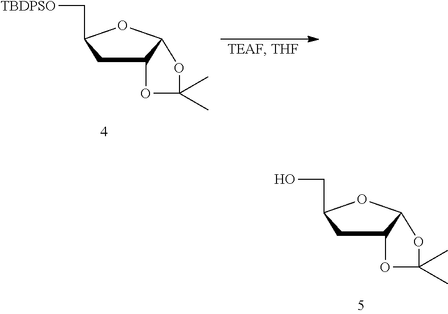

Compound 4 (35.0 g, 84.8 mmol) was dissolved in THF (500 mL) at 25° C. Tetraethylammonium fluoride (63.3 g, 424 mmol) was added, and the reaction mixture was stirred at 25° C. for 18 h until TLC (PE/EA=10/1, DCM/MeOH=10/1) showed complete consumption of reactants and formation of new spots. The reaction mixture was concentrated under reduced pressure to obtain a crude product. The crude product was purified by MPLC (DCM/MeOH=I/O-10/1) to obtain compound 5 (12 g, yield 81.21%) as a white solid.

¹H NMR (400 MHz,CD;OD) δ5.79 (d, J=3.6 Hz, 1H), 4.77 (t, J=4.0 Hz, 1H), 4.21-4.28 (m, 1H), 3.70 (dd, J=12.0, 3.6 Hz, 1H), 3.54 (dd, =12.0, 4.8 Hz, I H), 1.95-2.02 (m, 1H), 1.74 (ddd, J=13.2, 10.8, 4.8 Hz, 1H), 1.46 (s, 3H), 1.30 (s. 3 H).

5. Preparation of Compound 6

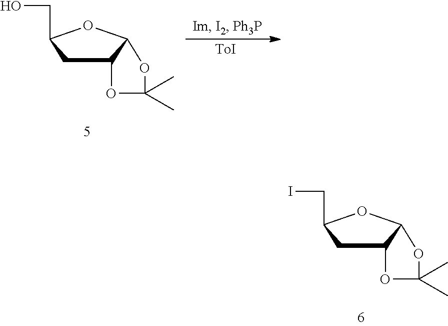

Compound 5 (12.0 g, 68.9 mmol) was dissolved in toluene (500 mL) at 25° C. Imidazole (9.38 g, 138 mmol), triphenylphosphine (36.1 g, 138 mmol), and elemental iodine (26.2 g, 103 mmol) were added in sequence. The reaction mixture was stirred at 100° C. for 3 h until TLC (PE/EA=5/1) showed complete consumption of reactants and formation of new spots. To the reaction mixture, 100 mL of saturated NaHSO₃ solution and 100 mL of water were added until the reaction mixture was separated into layers. The organic phase was washed with saturated NaCl solution (100 mL×3), dried over anhydrous Na₂SO₄, and then spin-dried to obtain a crude product. The crude product was purified by MPLC (PE/EA=I/O-10/1) to obtain compound 6 (16.6 g, yield 84.82%) as a white solid.

¹H NMR (400 MHz, CDCl₃) δ5.88 (d, J=3.6 Hz, 1H), 4.77 (t, J=4.2 Hz. 1 H), 4.13-4.21 (m, 1H), 3.25-3.38 (m, 2H), 2.31 (dd..J=13.6, 4.4 Hz, I H), 1.61-1.67 (m, I H), 1.52 (s, 3H), 1.33 (s, 3H).

6. Preparation of Compound 7

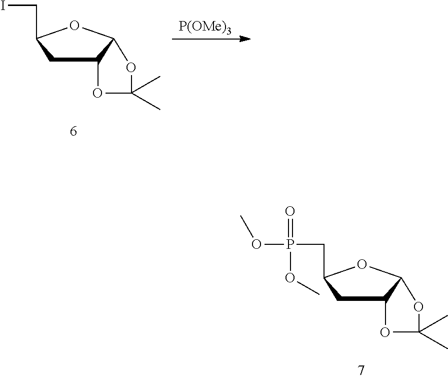

Compound 6 (40.0 g, 141 mmol) was dissolved in trimethyl phosphite (500 mL) at 25° C. The reaction mixture was stirred at 120° C. for 11 h until TLC (ethyl acetate/acetone=3/1, PE/EA=10/1) showed that the starting material remained and new spots were formed. The reaction mixture was spin-dried to obtain a crude product. The crude product was purified by MPLC (ethyl acetate/acetone=I/O-20/1) to obtain compound 7 (8.90 g, yield 23.74%) as a pale yellow oily liquid and recover starting material compound 7 (30.0 g) as a white solid.

¹H NMR (400 MHz, CDCl₃) δ 5.81 (d, J=4.0 Hz. 1 H), 4.74 (t, J=4.4 Hz, 1H), 4.40-4.52 (m, 1H), 3.76 (dd, J=10.8, 0.8 Hz, 6H). 2.23-2.35 (m, 2H), 1.95-2.07 (m, 1H), 1.63 (ddd, J=13.6, 10.8, 4.8 Hz, 1H), 1.52 (s, 3H), 1.32 (s, 3H).

7. Preparation of Compound 8

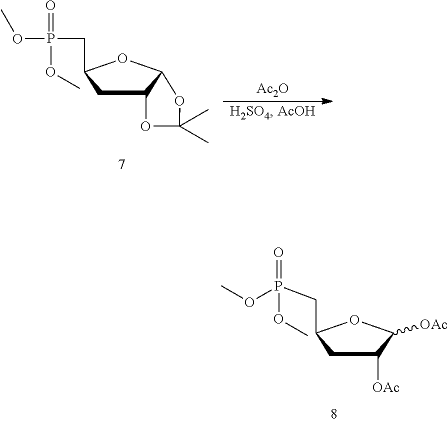

Compound 7 (13 g, 48.830 mmol), Ac₂O (23.036 mL, 244.150 mmol), and H₂SO₄ (2.615 mL, 48.830 mmol) were added in sequence to AcOH (260 mL). The reaction was allowed to proceed at 25° C. for 6 h until TLC (ethyl acetate:acetone=3:1) detected new points. The reaction mixture was quenched with ice water, extracted with DCM (500 mL×3), dried over anhydrous Na₂SO₄, filtered, and concentrated under reduced pressure to obtain a crude product. The crude product was purified by column chromatography (ethyl acetate:acetone=50:1-3:1) to obtain compound 8 (7.9 g, 25.464 mmol, 52.15%) as a yellow oil.

¹H NMR (400 MHz, CDCl₃) δ6.10 (d, J=1.2 Hz, 1H), 5.18 (d, J=5.2 Hz, 1H), 4.58-4.72 (m, 1H), 3.75-3.79 (m, 3H), 3.71-3.74 (m, 3H)2.16-2.38 (m, 3H), 2.07 (s, 3H), 2.05 (s, 3H), 1.96-2.04 (m, I H).

8. Preparation of Compound 9

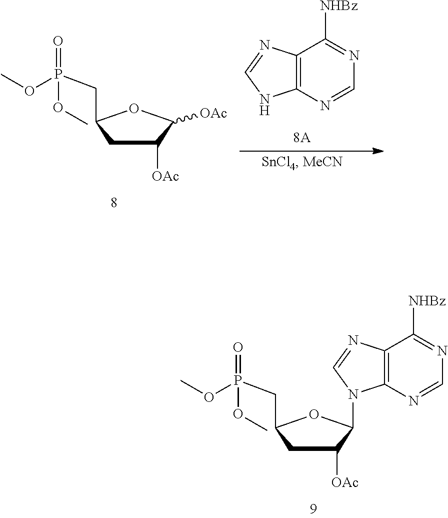

Compound 8 (7.9 g, 25.464 mmol) and compound 8A (6.09 g, 25.464 mmol) were dissolved in CH₃CN (316 mL), followed by the slow dropwise addition of SnCl4 (8.781 mL, 76.392 mmol) at 0° C. The reaction was allowed to proceed at 25° C. for 2 h until TLC (ethyl acetate:acetone=10:1, PMA) showed that the starting material compound 8 disappeared and new spots were formed and LCMS (RW0006-267-P1A) showed 31.3% product generation. The reaction mixture was cooled to 0° C. and adjusted to pH 8 with saturated aqueous NaHCO₃ solution. The aqueous phase was extracted with DCM (200 mL—3). The organic phase was dried over anhydrous Na₂SO₄, filtered, and spin-dried to obtain a crude product. The crude product was purified by column chromatography (ethyl acetate:acetone=20:1-3:1) to obtain compound 9 (6.1 g, 12.464 mmol, 48.95%) as a yellow oil.

¹H NMR (400 MHz, CDCl₃) δ 8.75-8.84 (m, 1H), 8.17 (s, 1H), 8.01-8.10 (m, 2H), 7.59-7.70 (m, 1 H), 7.50-7.57 (m, 2H), 6.07 (s, 1H), 5.66-5.73 (m, 1H), 4.98-5.12 (m, 1H), 4.70-4.81 (m, 1H), 3.71-3.82 (m, 12H), 2.70-2.81 (m, I H), 2.34-2.52 (m, 2H), 2.21-2.27 (m, I H), 2.16 (s, 3H), 2.06 (d, J=4.4 Hz, 4H).

9. Preparation of Compound 10

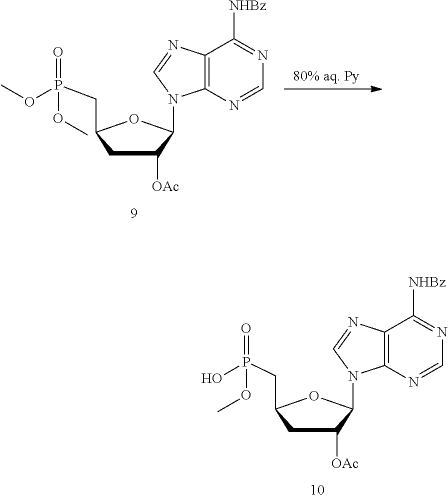

Compound 9 (6.1 g, 12.259 mmol) was dissolved in pyridine (30 mL) and water (20 mL). The reaction was allowed to proceed at 60° C. for 12 h until TLC (DCM:MeOH=10:1) showed that the starting material disappeared. The reaction mixture was spin-dried to obtain a crude product. The crude product was purified by Prep-HPLC (MeCN/H:O=30/1-80/1; flow rate: 30 mL/min) to obtain compound 10 (3.85 g, 8.098 mmol, 66.06%) as a yellow oil.

10. Preparation of Compound 11

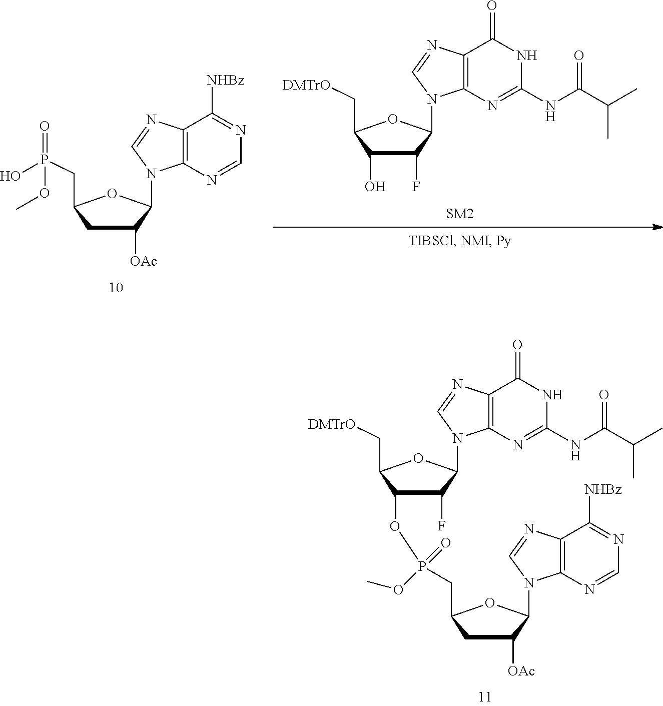

Compound 10 (3.7 g, 7.783 mmol) and compound SM2 (10.24 g, 15.566 mmol) were dissolved in pyridine (37 mL). 2,4,6-Triisopropylbenzenesulfonyl chloride (14.14 g, 46.698 mmol) was added at 0° C., and N-methylimidazole (5.11 g, 62.263 mmol) was slowly added dropwise to the reaction mixture after reaction for 60 min at 0° C. The reaction was allowed to proceed at 25° C. for 13 h until LCMS (RW0006-284-PlC) showed product generation and TLC (DCM:MeOH=10:1, UV) showed new points. The reaction mixture was cooled to 0° C., quenched with 50 mL of saturated sodium carbonate solution, and separated into layers. The organic phase was diluted with 300 mL of DCM, washed with water (50 mL×3) and saturated aqueous NaCl solution (50 mL×1), dried over anhydrous Na₂SO₄, filtered, and spin-dried to obtain a crude product. The crude product was purified by column chromatography (DCM:MeOH=2-4%, TEA) to obtain compound 11(2100 mg, 1.883 mmol, 24.20%) as a brown oil.

11. Preparation of Compound 12

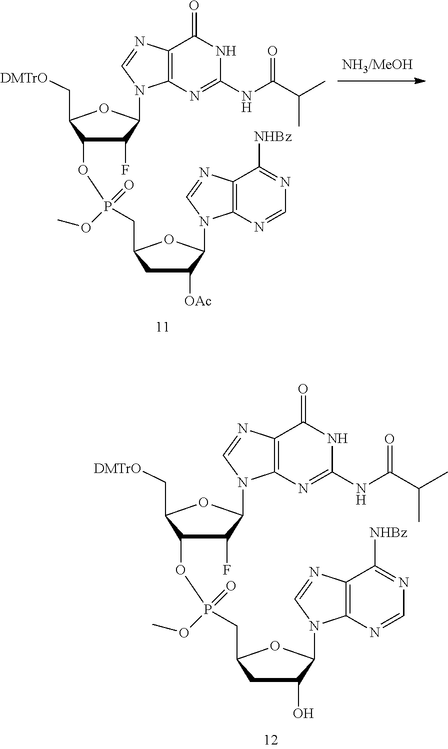

Compound 11 (2.4 g, 2.152 mmol) was dissolved in MeOH (36 mL). NH₃/MeOH (12.299 mL, 7M) was added and reacted at 0° C. for 3 h until LCMS (RW0006-290-P₁A) showed product generation. The reaction mixture was diluted with 150 mL of DCM and washed with 30 mL of water. The organic phase was dried over anhydrous Na₂SO₄, filtered, and spin-dried to obtain a crude product. The crude product was purified by Prep-HPLC (column: 01-Waters Xbridge BEH C_{18 19*150} mm; mobile phase: TEAA-ACN; gradient: 33%-58.5%/15 min, flow rate: 15 mL/min; ) to obtain compound 12 (380 mg, 0.354 mmol, 16.45%) as a yellow solid.

12. Preparation of E1

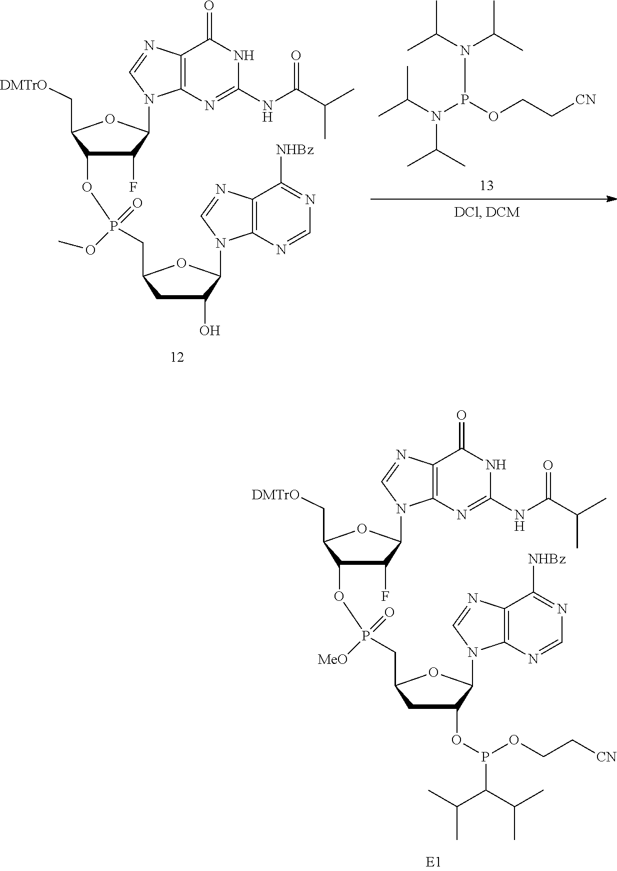

Compound 12 (380 mg, 0.354 mmol) was washed three times with acetonitrile, and dissolved in DCM (6.0 mL). 5A molecular sieve, DCI (41.82 mg, 0.354 mmol), and compound 13 (426.95 mg, 1.417 mmol) were added in sequence. The mixture was replaced with nitrogen three times and allowed to react at 25° C. for I h until LCMS (RW00006-291-PlA) showed that 5.4% of the starting material remained. The reaction mixture was quenched with saturated aqueous NaHCO₃ solution containing 20 mL of ice, diluted with 30 mL of DCM, and separated into layers. The organic phase was washed with 20 mL of saturated aqueous NaHCO₃ solution and 20 mL of saturated aqueous NaCl solution. The organic phase was dried over anhydrous Na₂SO₄, filtered, and spin-dried to obtain a crude product. The crude product was purified by reversed-phase chromatography (acetonitrile/water; 20-80%, 30 min, 20 mL/min) to obtain El (210 mg, 0.165 mmol, 46.57%) as a yellow solid.

¹H NMR (400 MHz, CDCl₃) δ 11.96-12.03 (m, 1H), 10.07-10.20 (m, 1H), 8.99-9.12 (m, 1H), 8.62-8.80 (m, 1H), 8.07-8.16 (m, 1H), 8.01-8.05 (m, 2H), 7.78 (d, .,=1.2 Hz, I H), 7.60-7.66 (m, I H), 7.51-7.58 (m, 2H), 7.21-7.26 (m, 2H), 7.09-7.20 (m, 7H), 6.66-6.75 (m, 4H), 6.37-6.56 (m, 1H), 5.75-6.11 (m, 3H), 5.07-5.19 (m, 1H), 4.60-4.74 (m, 1H), 4.16-4.29 (m, 1H), 3.81-3.89 (m, 1H), 3.76-3.81 (m, 3 H), 3.70 (d, J=1.6 Hz, 7H), 3.54-3.69 (m, 3H), 3.05-3.15 (m, 1H), 2.54-2.65 (m, 2H), 2.03-2.53 (m, 5H), 1.43-1.50 (m, I H), 1.14-1.21 (m, 11H), 1.01-1.13 (m, 6H).

### Example 2 Preparation of siRNA

The siRNA of the present invention was prepared using the solid-phase phosphoramidite method well known in the art. The specific methods can be referred to, for example, PCT Publication Nos. WO2016081444 and WO2019105419, and are briefly described below.

1 Synthesis of Sense Strand (SS)

Using the solid-phase phosphoramidite method, a blank CPG solid support or solid support conjugated with L₉₆ was used as the starting cycle, and nucleoside monomers were linked one by one according to the arrangement of sense strand nucleotides from the 3′ to 5′ direction. Each linkage of a nucleoside monomer involved four steps of deprotection, coupling, capping, and oxidation or thiolation to synthesize 5 pmol oligonucleotide with the synthesis conditions as follows:

Nucleoside monomers were provided in a 0.05 mol/L acetonitrile solution. The conditions for each step were identical: 25° C.; deprotection for 3 times using a 3% trichloroacetic acid-dichloromethane solution; coupling twice using a 0.25 mol/L ETT-acetonitrile solution as an activator; capping twice using 10% acetic anhydride-acetonitrile and pyridine/N-methylimidazole/acetonitrile (10:14:76, v/v/v); oxidation twice using 0.05 mol/L of iodine in tetrahydrofuran/pyridine/water (70:20:10, v/v/v); thiolation twice using 0.2 mol/L PADS in acetonitrile/3-methylpyridine (1:1, v/v).

2 Synthesis of Antisense Strand (AS)

Using the solid-phase phosphoramidite method, a blank CPG solid support was used as the starting cycle, and nucleoside monomers or nucleotide dimers of the present invention were linked one by one according to the arrangement of antisense strand nucleotides from the 3′ to 5′ direction. Each linkage of a nucleoside monomer or a nucleotide dimer of the present invention involved four steps of deprotection, coupling, capping, and oxidation or thiolation. The conditions for the synthesis of a 5 pmol oligonucleotide for the antisense strand were identical to those for the sense strand.

3 Purification and Annealing of Oligonucleotides

3.1 Ammonolvsis

The synthesized solid support (sense or antisense strand) was transferred to a 5 mL centrifuge tube, followed by the addition of 3% diethylamine/ammonia (v/v). The mixture was allowed to react in a thermostatic water bath at 35° C. (or 55° C.) for 16 h (or 8 h), and then filtered. The solid support was washed three times with ethanol/water, I mL each time. The filtrate was concentrated by centrifugation, and the crude product was purified.

3.2 Purification

The methods for purification and desalting are well known to those skilled in the art. For example, a strong anionic packing column can be used; a sodium chloride-sodium hydroxide system can be used for elution and purification. The product can be collected in tubes and desalted using a gel packing purification column. The elution system can be pure water.

3.3 Annealing

The sense strand (SS) was mixed with the antisense strand (AS) at a molar ratio (SS/AS=1/1.05) according to Table 6. The mixture was heated in a water bath to 70-95° C. for 3-5 min, and then allowed to cool naturally to room temperature. The system was freeze-dried to obtain the product.

The siRNA sequences used in the present invention as are follows:

| | Sequence (5′→3′) |
|---|---|
| Double | (The first one is the sense strand, |
| Strand | and the second one is the antisense |
| No. | strand.) |
| DR005709 | UmsGmsAmCmAmAmGfAfAfUmCmCmUmCmAmCmAmAm |
| | Um (SEQ ID NO: 1) |
| | AmsUfsUmGfUmGfAmGfGmAfUmUfCmUfUmGfUmCf |
| | AmsAfsCm (SEQ ID NO: 2) |
| DR005736 | UmsGmsAmCmAmAmGfAfAfUmCmCmUmCmAmCmAmAm |
| | Um (SEQ ID NO: 1) |
| | AmsUfsUmGfUmGf (ROR14-A) GfGmAfUmUfCm |
| | UfUmGfUmCfAmsAfsCm (SEQ ID NO: 3) |
| DR000359 | UmsUmsCmUmCmAmGfUfGfCmUmCmUmCmCmUmAmCm |
| | Um (SEQ ID NO: 4) |
| | AmsGfsUmAfGmGfAmGfAmGfCmAfCmUfGmAfGmAf |
| | AmsUfsUm (SEQ ID NO: 5) |
| DR005746 | UmsUmsCmUmCmAmGfUfGfCmUmCmUmCmCmUmAmCm |
| | Um (SEQ ID NO: 4) |
| | AmsGfsUmAfGmGf (ROR14-A) GfAmGfCmAfCm |
| | UfGmAfGmAfAmsUfsUm (SEQ ID NO: 6) |
| DR002220 | CmsAmsGmUmGfUmUfCfUfUmGmCmUmCmUmAmUmAm |
| | Am-L96 (SEQ ID NO: 7) |
| | UmsUfsAmUmAmGfAmGmCmAmAmGmAmAfCmAfCmUm |
| | GmsUmsUm (SEQ ID NO: 8) |
| DR005759 | CmsAmsGmUmGfUmUfCfUfUmGmCmUmCmUmAmUmAm |
| | Am-L96 (SEQ ID NO: 7) |
| | UmsUfsAmUmAmGf (ROR14-A) GmCmAmAmGmAm |
| | AfCmAfCmUmGmsUmsUm (SEQ ID NO: 9) |

The abbreviations used herein have the meaning as follows: PP8,N
A, U, G, and C represent anatural adenine ribonucleotide, a uracil ribonucleotide, a guanine ribonuclcotide, and a cytosine ribonucleotide, respectively.
m represents that the left nucleotide is a 2′-OCH₃ modified nucleotide. For example. Am, Um, Gmn, and Cm represent 2′-OCH₃ modified A, U, G, and C, respectively.
f represents that the left nucleotide is a 2′-F modified nucleotide. For example, Af, Uf, Gf, and Cf represent 2′-F modif ied A, U, G, and C, respectively.
“s” or “s-” represents that the flanking two nucleotides and/or delivery moiety are linked by a phosphorothioate group.

L₉₆ represents a GalNAc delivery moiety of the following structure well known in the art, wherein

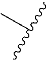

represents a position linked to siRNA by a phosphate group or a phosphorothioate group. See, for example. PCT Publication Nos. WO2009073809 and WO2009082607.

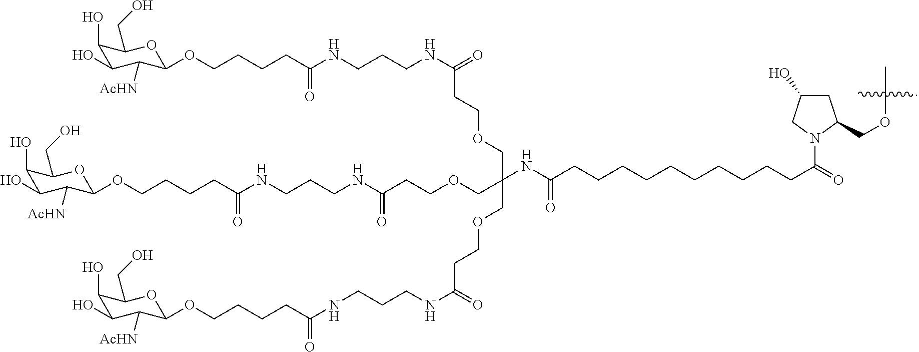 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

ROR14 represents a nucleotide substitute of the structure described above, wherein Base can be any base. For example, RORI4-A represents that tbc Base is adenine.

Specifically, the structure ofROR14-A is as follows:

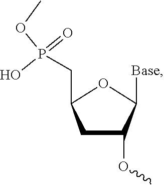

wherein Base is adenine.

### Example 3 Detection of On-target and Off-target Activity ofsiRNA Compound psi-CHECK2

1. Plasmid Preparation

On-target plasmid: The corresponding antisense strand on-target plasmid was designed according to the compound sequence. The recombinant plasmid psiCHECK2 GSCM was prepared by Sangon Biotech (Shanghai) Co., Ltd. and diluted to 1,000 ng/μL for later use.

Off-target plasmid: The corresponding antisense strand off-target plasmid was designed according to the compound sequence. The recombinant plasmid psiCHECK2 GSSM-5 Hits was prepared by Sangon Biotech (Shanghai) Co., Ltd. and diluted to 1,000 ng/pL for later use.

2. Cell Transfection

HEK293A cells (Nanjing Cobioer Biotechnology Co., Ltd., Cat #: CBP60436) were used, and 100 μL of cell suspension was seeded into a 96-well plate at 8x 10; cells/well.

On the following day, the complete medium in the wells was discarded and replaced with Opti-MEM medium at 80 μL/well. The cells were starved for 1.5 h.

Plasmid mixture: plasmid: 0.01 pL/well, Opti-MEM: 8.99 pL/vell.

Lipo mixture: Lipo 2000 (LipofectamineTh 2000 transfection reagent. Thermo, 11668019) was diluted with Opti-MEM and allowed to stand at room temperature for 5 min. The Lipo mixture was prepared as follows: Lipo; 0.2 μL/well. Opti-MEM: 9.8 μL/well.

Subsequently, 22 μL of the prepared Lipo mixture, 2.2 μL of compound, and 19.8 μL of plasmid mixture were loaded into the same well (designated as Well A), mixed well by pipetting up and down, and incubated at room temperature for 20 min before co-transfection. Well A mixture was then added to each well of cells at 20 μL/well to obtain a final volume of 100 μL/well (20 μL of Well A mixture+existing 80 μL of Opti-MEM). After incubation in a 5% CO₂ incubator at 37° C. for 4 h, 100 μL of DMEM containing 20% fetal bovine serum was added to each well. Testing was performed after further incubation in a 5% CO₂ incubator at 37° C. for 24 h.

3. Testing

The mixed Dual-Glo® Luciferase (Dual-Glo® Luciferase Assay System, Promega. E2940) was thawed and equilibrated to room temperature before testing. DMEM was then added to each tube at a 1:1 ratio to prepare substrate I immediately before use. Similarly, the Dual-Glo® Stop & Glo® Buffer was thawed, equilibrated to room temperature, and then mixed with the Dual-Glo® Stop & Glo® Substrate at a 100:1 ratio to prepare substrate 11 immediately before use.

The existing culture medium in the 96-well plate was aspirated using a vacuum pump;

150 μL of substrate I was added to each well and incubated on a shaker at room temperature for 10 min:

120 μL of substrate I was transferred to a 96-well microplate and the Firefly chemiluminescence value was read on a microplate reader (Tecan, Infinite 200);

Another 60 μL of substrate II was added to each well and incubated on a shaker at room temperature for 10 min. The Renilla chemiluminescence value was read on a microplate reader.

4. Data Analysis and Processing

The fluorescence activity was measured using a microplate reader. The collected Renilla signal was normalized to the Firefly signal standard. The inhibitory effect of siRNA was determined through comparison with unprocessed results (residual inhibitory activity). The calculation process is as follows:

Normalized Ren/Fir ratio: Ratio=Renilla (Renilla luciferase)/Firefly (Firefly luciferase).

Residual inhibition: (Ratios.RN_{A}/Ratioc,nₘI)*100%, taking the mean of two replicate wells, where Ratio,o˜_{d}o, was the mean ratio value of two replicate control wells (without siNRA). The Ratio˜,A/Ratiocontr.1 was calculated for each of the two replicate wells, and the mean of these two values was taken as the residual inhibition.

Plotting: Graphpad Prism was used for plotting.

Half maximal inhibitory concentration (IC₅₀): Top and Bottom were plotted in this experiment. The IC₅₀ value was calculated according to the formula Y=Bottom+(Top-Bottom)/(1+10\hat{ }((LogIC₅₀-X)*HillSlope)), where Y=50 and X=log(concentration).

In the on-target activity test, the HEK293A cell line (Nanjing Cobioer Biotechnology Co., Ltd., Cat #; CBP60436) was used for transfection with the psiCHECK2-GSCM recombinant plasmid. The starting concentration of the compound was 10 nM, and 11 concentration points (10 nM, 3.33 nM, 1.11 nM, 0.37 nM, 0.123 nM, 0.041 nM, 0.0136 nM, 0.0045 nM, 0.00152 nM, 0.000508 nM, 0.000169 nM) were obtained by serial 3-fold dilutions for siRNA compound activity screening. The results are shown in Table 1.

TABLE 1
Test Results of On-target Activity of psi-CHECK2

| | Double | | | | | | |
|---|---|---|---|---|---|---|---|
| | Strand | | | | | | |
| Gene | Code | 10 nM | 3.3333 nM | 1.1111 nM | 0.37037 nM | 0.12346 nM | 0.04115 nM |
| HBV-S | DR005709 | 1.5% | 1.8% | 1.8% | 2.3% | 6.8% | 20.3% |
| | DR005736 | 2.0% | 2.0% | 1.8% | 2.1% | 3.6% | 11.0% |
| APOC3 | DR000359 | 0.8% | 0.7% | 0.8% | 1.1% | 3.5% | 12.7% |
| | DR005746 | 1.1% | 0.9% | 1.0% | 1.3% | 4.1% | 14.1% |

| | Double | | | | | | |
|---|---|---|---|---|---|---|---|
| | Strand | | | | | | IC₅₀ |
| Gene | Code | 0.01372 nM | 0.004570M | 0.00152 nM | 0.00051 nM | 0.00017 nM | (nM) |
| HBV-S | DR005709 | 53.4% | 82.4% | 96.6% | 105.2% | 97.0% | 0.0142 |
| | DR005736 | 39.3 | 76.5% | 107.8% | 122.0% | 121.9% | 0.0067 |
| APOC3 | DR000359 | 37.0% | 72.7% | 94.4% | 99.4% | 99.5% | 0.0092 |
| | DR005746 | 38.3% | 73.9% | 95.2% | 104.6% | 99.6% | 0.0092 |

In the off-target activity test, the HEK293A cell line (Nanjing Cobioer Biotechnology Co., Ltd., Cat #.—CBP60436) w˜as used for transfection with the psiCHECK2-GSSM-5 Hits recombinant plasmid. The starting concentration of the compound was 10 nM, and 11 concentration points (10 niM, 3.33 nM, 1.11 nM, 0.37 nM, 0.123 nM, 0.041 nM, 0.0136 nM, 0.0045 nM, 0.00152 nM, 0.000508 nM, 0.000169 nM) were obtained by serial 3-old dilutions for siRNA compound activity screening. The results are shown in Table 2.

TABLE 2
Test Results of Off-target Activity of psi-CHECK2

| | Double | | | | | | | |
|---|---|---|---|---|---|---|---|---|
| | Strand | | | | | | | |
| Gene | Code | 10 nM | 3.3333 nM | 1.111 nM | 0.37037 nM | 0.12346 nM | 0.04115 nM | 0.01372 nM |
| HBV-S | DR005709 | 3.1% | 4.0% | 4.9% | 15.6% | 60.8% | 100.2% | 115.4% |
| | DR005736 | 633% | 75.7 | 94.3% | 105.0% | 109.1% | 106.2% | 110.2% |
| APOC3 | DR000359 | 9.5% | 8.8% | 10.4% | 20.4% | 53.8% | 91.7% | 98.6% |
| | DR005746 | 545% | 58.4% | 73.6% | 88.3% | 100.0% | 97.5% | 103.6% |

| | | | | | | Off- |
|---|---|---|---|---|---|---|
| | Double | | | | | target |
| | Strand | | | | | IC₅₀ |
| Gene | Code | 0.00457 nM | 0.00152 nM | 0.00051 nM | 0.00017 nM | (nM) |
| HBV-S | DR005709 | 110.2 | 110.9% | 113.5% | 108.7% | 0.013 |
| | DR005736 | 105.9% | 108.5 | 108.4% | 100.8% | >10 |
| APOC3 | DR000359 | 105.3% | 104.1% | 106.8% | 101.6% | 0.117 |
| | DR005746 | 103.2% | 106.1 | 104.4% | 99.0% | >10 |

The results showed that siRNA carring ROR14 effectively reduced off-target activity while maintaining on-target activity.

### Example 4 Long-term Pharmacodynamic Validation of Compounds in C₅₇BL/6 Mouse Model

C57BL/6 mice (male. 18-21 g, 6-8 weeks old) were randomized into 6 animals per group according to Table 3. The dose for each animal was calculated based on body weight and administered subcutaneously as a single dose. The siRNA compound was first prepared as a 1 mg/mL solution (0.9% aqueous sodium chloride solution as solvent), and then dissolved and diluted to the desired concentration and volume with 0.9% aqueous sodium chloride solution before the experiment. The dose volume was 5 mL,7g for both the normal saline and siRNA compound.

TABLE 3
Animal Grouping

| S/N | Compound-Group |
|---|---|
| 1 | Saline |
| 2 | DR002220 |
| 3 | DR005759 |

Blood samples were collected from the orbital venous plexus of mice before administration (with the day of administration designated as Day 0) and on Days 14, 28, 42, and 56 after administration to detect serum mTTR protein using an ELISA kit (Abcam, ab282297) at each time point; 10 mg of liver was extracted and placed in RNAlater solution at the last experimental time point to detect mTTR mRNA in the liver.

TABLE 4
Long-term Pharmacodynamic Validation Results
of Compounds in C57BL/6 Mouse Model

| | Saline | | DR002220 | | DR005759 | |
|---|---|---|---|---|---|---|
| | Residual mTTR | | Residual mTTR | | Residual mTTR | |
| | Protein | SD | Protein | SD | Protein | SD |
| Day 0 | 100% | 20% | 101% | 10% | 100% | 22% |
| Day 14 | 103% | 7% | 9% | 3% | 6% | 1% |
| Day 28 | 120% | 7% | 17% | 4% | 19% | 8% |
| Day 42 | 124% | 6% | 37% | 11% | 46% | 13% |
| Day 56 | 127% | 10% | 62% | 10% | 72% | 11% |

The results showed that siRNA carring ROR14 reduced target gene expression in vivo for a long time.

1. A nucleotide dimer of formula (A): 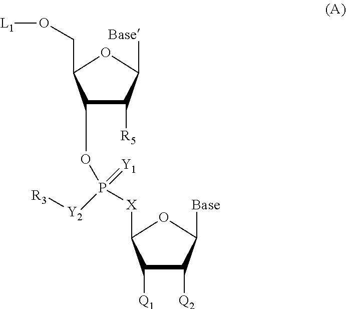
wherein one of Q₁ and Q₂ is R₄, and the other is O-L₂;
L₁ is H or P₁, or a bond to the phosphorus atom P of the phosphate group at the 2 or 3′ end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂;
X is selected from —(CR₁R₂)ₘ—or —CR₁αCR₂—;
Y₁ is O, S, or NR;
Y₂ is O, S, or a bond;
R₁ and R₂ are independently selected from H, D, halogen, OH, CN, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl, which are optionally substituted with 1, 2, 3, 4, 5, 6, 7, 8, or more R;
R₃ is selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₆₋₁₀ aryl, or 5-membered to 14-membered heteroaryl, which are optionally substituted with 1, 2, 3, 4, 5, 6, 7, 8, or more R′;
R₄ and R₅ are independently selected from H, D, OH, halogen, C₁₋₆ alkyl, C₁₋₆ haloalkyl, or C₁₋₆ alkoxyl, preferably H, F, or OMe.
P₁ is a hydroxy-protecting group, preferably DMTr;
P₂ is a reactive phosphorus group, preferably —P(OCH₂CH₂CN)(N(iPr)₂);
Base and Base′ are independently selected from H, a modified or unmodified base or leaving group, preferably modified or unmodified A, U, T, G, and C;
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, Ce₆₋₁₀ aryl, 5-membered to 14-membered heteroaryl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —S(O)ₙR^{a}, —S(O)ₙOR^{a}, —S(O)ₙNR^{a}R^{b}, —O—S(O)ₙR^{b}, —NR^{a}R^{b}, —NR^{a}C(O)R^{b}, —NR^{a}—C(O)OR^{b}, —NR^{a}—S(O)ₙR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, C₂₋₆ alkynyl, C₃₋₁₀ cycloalkyl, 3-membered to 10-membered heterocyclyl, C₁₋₁₀ aryl, or 5-membered to 14-membered heteroaryl; or R^{a} and R^{b} and the nitrogen atom to which they are attached form a 3-membered to 10-membered heterocyclyl;
m is selected from 1, 2, 3, 4 or 5;
n is independently selected from 1 or 2.

2. A nucleotide dimer according to claim 1, wherein the nucleotide dimer has a structure of formula (I) or (II): 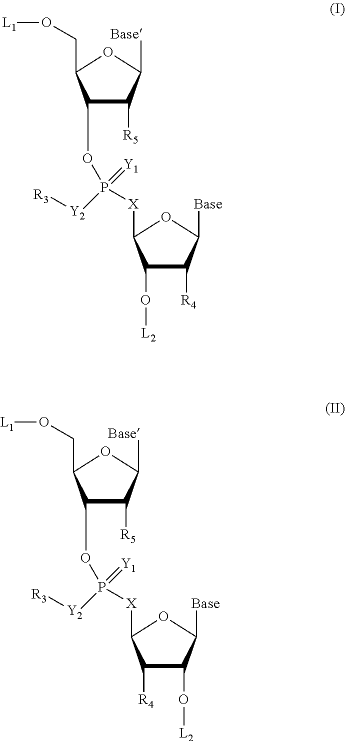
wherein each group is as defined in claim 1.

3. A nucleotide dimer according to claim 2, wherein the nucleotide dimer has one or more of the following definitions:
i) wherein,
X is selected from —(CR₁R₂)ₘ—or —CR₁=CR₂—;
R₁ and R₂ are independently selected from H, D, halogen, OH, CN, C₁₋₆ alkyl, C₁₋₆ haloalkyl, C₂₋₆ alkenyl, or C₂₋₆ alkynyl, which are optionally substituted with 1, 2, 3, 4, 5, or more R′;
R′ is selected from D, halogen, CN, C₁₋₆ alkyl, C₁₋₆ haloalkyl, C₂₋₆ alkenyl, C₂₋₆ alkynyl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —NR^{a}R^{b}, —NR^{a}C(O)R^{b}, —NR^{a}—C(O)OR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, or C₂₋₆ alkynyl;
m is selected from 1, 2, 3, 4 or 5;
preferably,
X is selected from —(CR₁R₂)ₘ—or —CR₁═CR₂—;
R₁ and R₂ are independently selected from H, D, halogen, OH, CN, C₁₋₆ alkyl, or C₁₋₆ haloalkyl, which are optionally substituted with 1, 2, 3, or more R′;
R′ is selected from D, halogen, CN, C₁₋₆ alkyl, C₁₋₆ haloalkyl, C₂₋₆ alkenyl, C₂₋₆ alkynyl, —OR^{a}, or —NR^{a}R^{b};
R^{a} and R^{b} are independently selected from H, C₁₋₆ alkyl, or C₁₋₆ haloalkyl;
m is selected from 1, 2, or 3;
more preferably,
X is selected from —CH₂—, —CH(OH)—, —CH₂—CH₂—, or —CH═CH;
ii) wherein,
Y₁ is O or NR;
Y₂ is O, S, or a bond:
R is selected from H, C₁₋₆ alkyl, or C₁₋₆ haloalkyl;
preferably,
Y₁ is O or NR;
Y₂ is O, S, or a bond;
R is selected from H or C₁₋₆ alkyl;
more preferably,
Y₁ is O;
Y is O;
iii) wherein,
R₃ is selected from H, C,₁₋₆ alkyl, C₁₋₆ haloalkyl, C₂₋₆ alkenyl, or C₂₋₆ alkynl, which are optionally substituted with 1, 2, 3, 4, 5, r more R′;
preferably,
R₃ is selected from H, C₁₋₆ alkyl, or C₁₋₆ haloalkyl, which is optionally substituted with 1, 2, 3, or more R′;
more preferably,
R₃ is C₁₋₄ alkyl, preferably Me;
iv) wherein,
R₄ and R₅ are independently selected from H, D, OH, halogen, C₁₋₆ alkyl, C₁₋₆ haloalkyl, or C₁₋₆ alkoxyl;
preferably,
R₄ and R₅ are independently selected from H, OH, halogen, C₁₋₆ alkyl, C₁₋₆ haloalkyl, or C₁₋₆ alkoxyl, preferably H, F, OH, or QMe more preferably,
R₄ is selected from H, OH, or C₁₋₄ alkoxyl, preferably H or OMe;
R₅ is selected from haloae, OH, or C₁₋₄ alkoxyl, preferably F or Ome;
v) wherein,
Base and Base′ are independently selected from 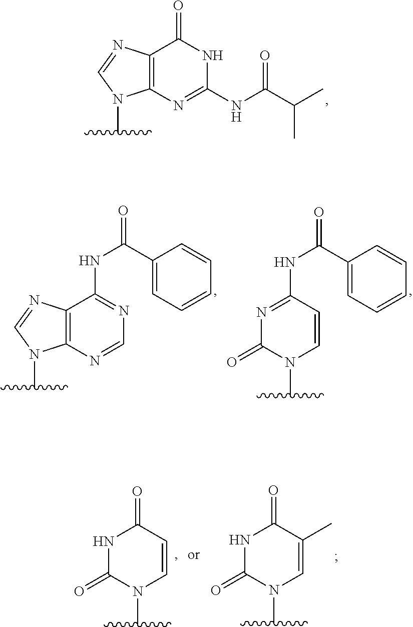
vi) wherein,
L₁ is H or P₁, or a bond to the phosphorus atom P of the phosphate group at the 2 or Y end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂;
X is selected from —(CR₁R₂)ₘ—or —CR₁═CR₂—;
Y₁ is O, S, or NR;
Y₂ is O, S, or a bond;
R₁ and R₂ are independently selected from H, D, halogen, OH, CN, C₁₋₆ alkyl, C₁₋₆ haloalkyl, C₂₋₆ alkenyl, or C₂₋₆ alkynyl, which are optionally substituted with 1, 2, 3, 4, 5, or more R′:
R₃ is selected from H, C₁₋₆ alkyl, C₁₋₆ haloalkyl, C₂₋₆ alkenyl, or C₂₋₆ alkynyl, which are optionally substituted with 1.2, 3.4, 5, or more R′:
R₄ and R₅ are independently selected from H, D, OH, halogen, C₁₋₆ alkyl, C₁₋₆ haloalkyl, or C₁₋₆ alkoxyl, preferably H, F, or OMe:
P₁ is a hydroxy-protecting group, preferably DMTr:
P₁₂ is a reactive phosphorus group, preferably —P(OCH₂CH₂CN)(N(iPr)_{b});
Base and Base′ are independently selected from H, a modified or unmodified base or leaving group, preferably modified or unmodified A, U, T, G, and C:
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, —OR^{a}, —OC(O)R^{a}, —O—C(O)OR^{b}, —O—C(O)NR^{a}R^{b}, —C(O)R^{a}, —C(O)OR^{a}, —C(O)NR^{a}R^{b}, —NR^{a}R^{b}, —NR^{a}C(O)R^{b}, —NR^{a}—C(O)OR^{b}, or —NR^{a}C(O)NR^{a}R^{b};
R^{a} and R^{b} are independently selected from H, C₁₋₆ alkyl, C₁₋₆ haloalkyl, C₂₋₆ alkenyl, or C₂₋₆ alkynyl;
m is selected from 1, 2, 3, 4, or 5; and
vii) wherein,
L₁ is H or P₁, or a bond to the phosphorus atom P of the phosphate group at the 2 ′ or 3 ′ end of the ribose of another nucleotide or oligonucleotide, preferably P₁;
L₂ is H or P₁, or a bond to the phosphorus atom P of the phosphate group at the 5′ end of the ribose of another nucleotide or oligonucleotide, preferably P₂;
X is selected from —(CR₁R₂)ₘ—or —CR₁═CR₂—:
Y₁ is O or NR;
Y₂ is O, S, or a bond;
R₁ and R₂ are independently selected from H, D, haloaen, OH, CN, C₁₋₆ alkyl, or C₁₋₆ haloalkyl, which are optionally substituted with 1, 2, 3, or more R′;
R₃ is selected from H, C₁₋₆ alkyl, or C₁₋₆ haloalkyl, which is optionally substituted with 1, 2, 3, or more R′;
R₄ and R₅ are independently selected from H, D, halogen, C₁₋₆ alkyl, C₁₋₆ haloalkyl, or C₁₋₆ alkoxyl, preferably H, F, or OMe;
P₁ is a hydroxy-protecting group, preferably DMTr:
P₂ is a reactive phosphorus group, preferably —P(OCH₂CH₂CN)(N(iPr)₂);
Base and Base′ are independently selected from H, a modified or unmodified base or leaving group, preferably modified or unmodified A, U, T, G, and C;
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, —OR^{a}, or —NR^{a}R^{b};
R^{a} and R^{b} are independently selected from H, C₁₋₆ alkyl, or C₁₋₆ haloalkyl;
m is selected from 1, 2,r 3.

4-9. (canceled)

10. A nucleotide dimer according to claim 2 wherein, L₁ is H or P₁, or a bond to the phosphorus atom P of the phosphate group at the 2′ or 3′ end of the ribose of another nucleotide or oligonucleotide, preferably P₁;
L₂ is H or P₂, or a bond to the phosphorus atom P of the phosphate group at the 5′ end of the ribose of another nucleotide or oligonucleotide, preferably P₂;
X is selected from —(CR₁R₂)ₘ—or —CR₁=CR₂—, preferably —CH₂—, —CH(OH)—, —CH₂—CH₂—, or —CH═CH—;
Y₁ is O;
Y₂ is O;
R₁ and R₂ are independently selected from H, D, halogen, OH, CN, C₁₋₄ alkyl, or C₁₋₄ haloalkyl;
R₃ is C₁₋₄ alkyl, preferably Me;
R₄ is selected from H, OH, or C₁₋₄ alkoxyl, preferably H or OMe;
R₅ is selected from halogen, OH, or C₁₋₄ alkoxyl, preferably F or OMe;
P₁ is a hydroxy-protecting group, preferably DMTr;
P₂ is a reactive phosphorus group, preferably —P(OCH₂CH₂CN)(N(iPr)₂); Base and Base′ are independently selected from 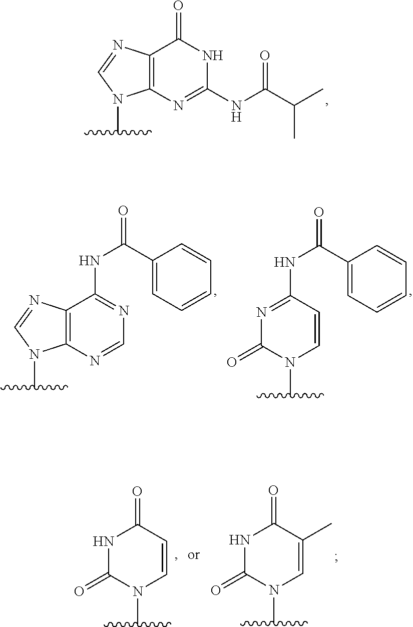
m is selected from 1, 2, or 3.

11. A nucleotide dimer according to claim 1, wherein the nucleotide dimer is selected from: 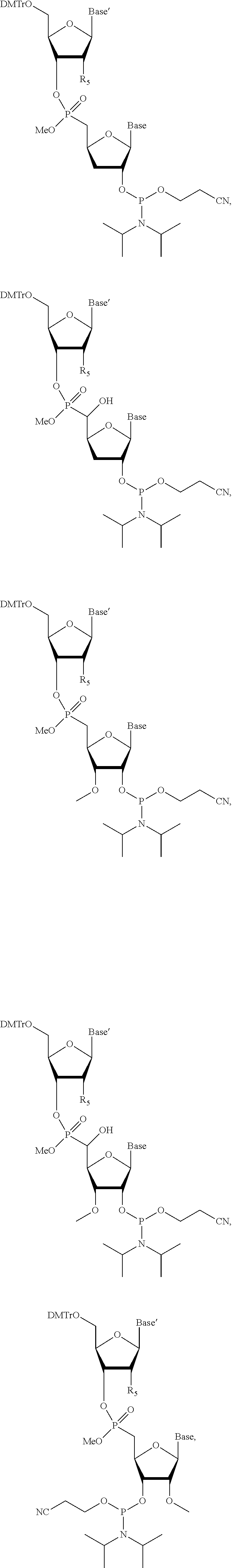 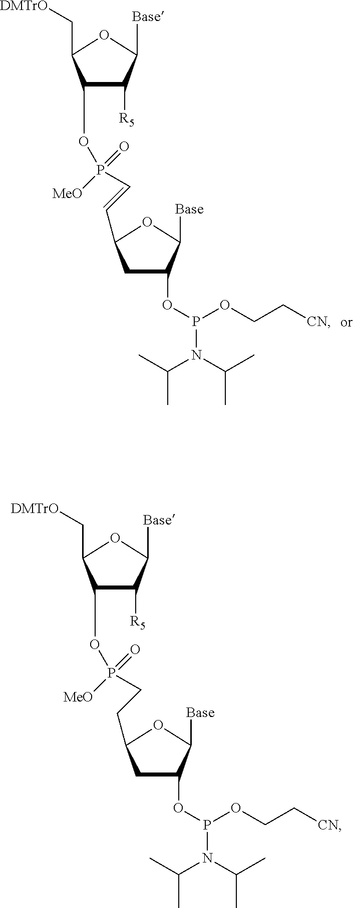
wherein Base and Base′ are as defined in claim 1, preferably 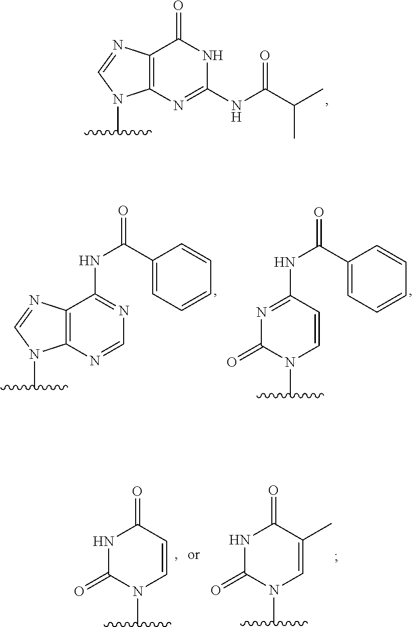
R5 is as defined in claim 1, preferably F or OMe.

12. A dsRNA molecule, or a pharmaceutically acceptable salt, tautomer, or stereoisomer thereof, comprising a sense strand and an antisense strand, each strand having 14 to 30 nucleotides;
wherein the antisense strand comprises one or more nucleotide monomers of formula (III) or (IV): 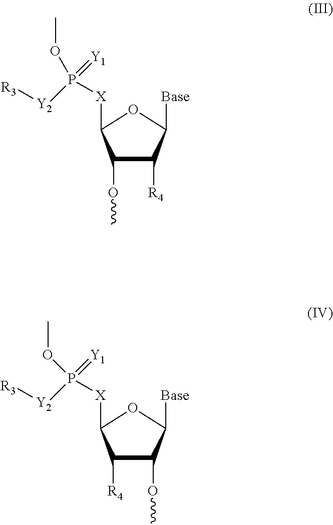
wherein the nucleotide monomers are connected from ----- to [symbol] in the 5′ to 3′ direction;
each group is as defined in claim 1;
preferably, the nucleotide monomers are selected from: 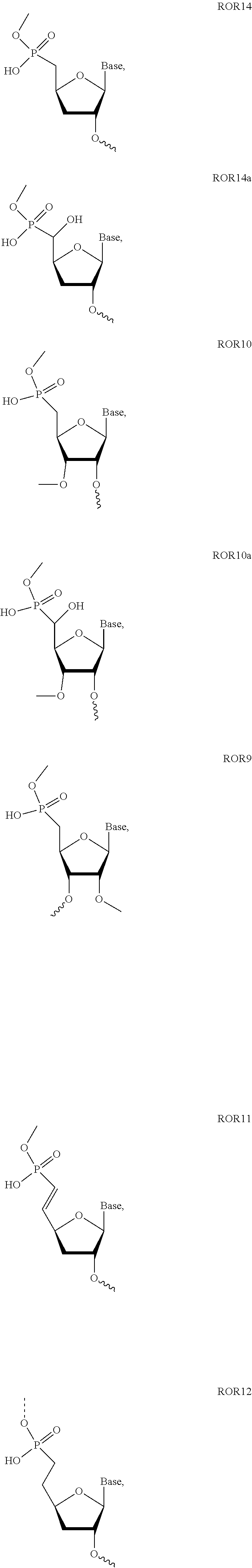
wherein Base is selected from

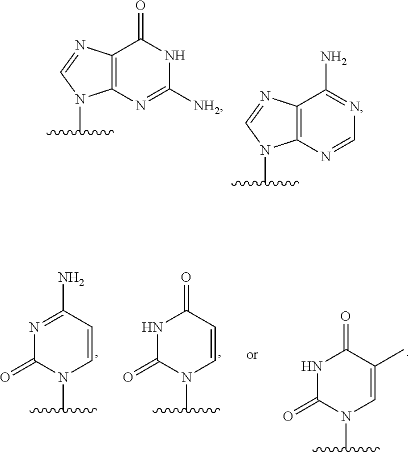

13. A dsRNA molecule according to claim 12, wherein the dsRNA molecule has one or more of the following definitions:
i) wherein the sense strand and the antisense strand each have 20 to 25 nucleotides;
ii) wherein the nucleotide monomer is located at positions 2 to 8, preferably position 6 or 7, more preferably position 7, of the 5′ end of the antisense strand;
iii) wherein the dsRNA exhibits enhanced stability compared with a dsRNA having the same sequence but not comprising the nucleotide monomer of claim 12;
iv) wherein the dsRNA has a melting temperature of about 40° C. to about 80° C. preferably about 55° C. to 67° C.:
v) wherein the dsRNA exhibits reduced off-target toxicity compared with a dsRNA having the same sequence but not comprising the nucleotide monomer of claim 12;
vi) wherein the dsRNA exhibits enhanced effectiveness compared with a dsRNA having the same sequence but not comprising the nucleotide monomer of claim 12;
vii) wherein the antisense strand has a sequence sufficiently complementary to the sense strand and target mRNA and is capable of inducing degradation of the target mRNA;
viii) wherein the target mRNA is encoded by an endogenous genre or by a pathogen gene;
ix) wherein the sense strand and/or antisense strand comprises a 3′ and/or 5′ over-hang; and
x) wherein the dsRNA is further conjugated to a ligand; preferably, the ligand comprises one or more GalNAc.

14-22. (canceled)

23. A nucleic acid molecule comprising in its nucleotide sequence one or more nucleotide monomers of claim 12.

24. A nucleic acid molecule according to claim 23, wherein the nucleic acid is selected from DNA, RNA, and a DNA/RNA hybrid.

25. A nucleic acid molecule according to claim 24, wherein the nucleic acid molecule is single- or double-stranded.

26. A nucleic acid molecule according to claim 23, wherein the nucleic acid molecule is selected from small interfering RNA (siRNA) and short hairpin RNA (shRNA).

27. A vector comprising a nucleotide sequence encoding a dsRNA of claim 12.

28. A cell comprising a dsRNA of claim 12.

29. A pharmaceutical composition comprising the dsRNA molecule according to claim 12 and a pharmaceutically acceptable carrier or excipient,t.

30. A kit comprising the dsRNA molecule according to claim 12.

31. A method for inhibiting the expression of a target gene in a cell, comprising a step of introducing the dsRNA molecule according to claim 12 into the cell.

32. A method for inhibiting the expression of a target gene in a cell, comprising expressing the dsRNA molecule according to claim 12 in the cell.

33. A method for reducing off-target toxicity in a cell, comprising a step of introducing the dsRNA molecule according to claim 12 into the cell.

34. A method for reducing off-target toxicity in a cell, comprising expressing the dsRNA molecule according to claim 12 in the cell.

35. A cell comprising a vector of claim 27.


SEQUENCE LISTING (9 sequences)

SEQ ID NO: 1 - RNA, 19 nt, synthetic construct
  source 1..19: mol_type = other RNA
  misc_feature 1..19: note = sense strand
  modified_base 1..2: mod_base = OTHER; note = thio; note = 2'-O-methyl
  modified_base 3..6: mod_base = OTHER; note = 2'-O-methyl
  modified_base 7..9: mod_base = OTHER; note = 2'-fluoro
  modified_base 10..19: mod_base = OTHER; note = 2'-O-methyl
tgacaagaatcctcacaat

SEQ ID NO: 2 - RNA, 21 nt, synthetic construct
  source 1..21: mol_type = other RNA
  misc_feature 1..21: note = antisense strand
  modified_base 1: mod_base = OTHER; note = thio; note = 2'-O-methyl
  modified_base 2: mod_base = OTHER; note = thio; note = 2'-fluoro
  modified_base 3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = 2'-O-methyl
  modified_base 8: mod_base = OTHER; note = 2'-fluoro
  modified_base 9: mod_base = OTHER; note = 2'-O-methyl
  modified_base 10: mod_base = OTHER; note = 2'-fluoro
  modified_base 11: mod_base = OTHER; note = 2'-O-methyl
  modified_base 12: mod_base = OTHER; note = 2'-fluoro
  modified_base 13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17: mod_base = OTHER; note = 2'-O-methyl
  modified_base 18: mod_base = OTHER; note = 2'-fluoro
  modified_base 19: mod_base = OTHER; note = 2'-O-methyl; note = thio
  modified_base 20: mod_base = OTHER; note = 2'-fluoro; note = thio
  modified_base 21: mod_base = OTHER; note = 2'-O-methyl
attgtgaggattcttgtcaac

SEQ ID NO: 3 - RNA, 21 nt, synthetic construct
  source 1..21: mol_type = other RNA
  misc_feature 1..21: note = antisense strand
  modified_base 1: mod_base = OTHER; note = 2'-O-methyl; note = thio
  modified_base 2: mod_base = OTHER; note = 2'-fluoro; note = thio
  modified_base 3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = n is ROR14-A
  modified_base 8: mod_base = OTHER; note = 2'-fluoro
  modified_base 9: mod_base = OTHER; note = 2'-O-methyl
  modified_base 10: mod_base = OTHER; note = 2'-fluoro
  modified_base 11: mod_base = OTHER; note = 2'-O-methyl
  modified_base 12: mod_base = OTHER; note = 2'-fluoro
  modified_base 13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17: mod_base = OTHER; note = 2'-O-methyl
  modified_base 18: mod_base = OTHER; note = 2'-fluoro
  modified_base 19: mod_base = OTHER; note = 2'-O-methyl; note = thio
  modified_base 20: mod_base = OTHER; note = 2'-fluoro; note = thio
  modified_base 21: mod_base = OTHER; note = 2'-O-methyl
attgtgnggattcttgtcaac

SEQ ID NO: 4 - RNA, 19 nt, synthetic construct
  source 1..19: mol_type = other RNA
  modified_base 1..2: mod_base = OTHER; note = thio; note = 2'-O-methyl
  modified_base 3..6: mod_base = OTHER; note = 2'-O-methyl
  modified_base 7..9: mod_base = OTHER; note = 2'-fluoro
  modified_base 10..19: mod_base = OTHER; note = 2'-O-methyl
  misc_feature 1..19: note = sense strand
ttctcagtgctctcctact

SEQ ID NO: 5 - RNA, 21 nt, synthetic construct
  source 1..21: mol_type = other RNA
  modified_base 1: mod_base = OTHER; note = thio; note = 2'-O-methyl
  modified_base 2: mod_base = OTHER; note = 2'-fluoro; note = thio
  misc_feature 1..21: note = antisense strand
  modified_base 3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = 2'-O-methyl
  modified_base 8: mod_base = OTHER; note = 2'-fluoro
  modified_base 9: mod_base = OTHER; note = 2'-O-methyl
  modified_base 10: mod_base = OTHER; note = 2'-fluoro
  modified_base 11: mod_base = OTHER; note = 2'-O-methyl
  modified_base 12: mod_base = OTHER; note = 2'-fluoro
  modified_base 13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17: mod_base = OTHER; note = 2'-O-methyl
  modified_base 18: mod_base = OTHER; note = 2'-fluoro
  modified_base 19: mod_base = OTHER; note = thio; note = 2'-O-methyl
  modified_base 20: mod_base = OTHER; note = thio; note = 2'-fluoro
  modified_base 21: mod_base = OTHER; note = 2'-O-methyl
agtaggagagcactgagaatt

SEQ ID NO: 6 - RNA, 21 nt, synthetic construct
  source 1..21: mol_type = other RNA
  misc_feature 1..21: note = antisense strand
  modified_base 1: mod_base = OTHER; note = 2'-O-methyl; note = thio
  modified_base 2: mod_base = OTHER; note = thio; note = 2'-fluoro
  modified_base 3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = n is ROR14-A
  modified_base 8: mod_base = OTHER; note = 2'-fluoro
  modified_base 9: mod_base = OTHER; note = 2'-O-methyl
  modified_base 10: mod_base = OTHER; note = 2'-fluoro
  modified_base 11: mod_base = OTHER; note = 2'-O-methyl
  modified_base 12: mod_base = OTHER; note = 2'-fluoro
  modified_base 13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17: mod_base = OTHER; note = 2'-O-methyl
  modified_base 18: mod_base = OTHER; note = 2'-fluoro
  modified_base 19: mod_base = OTHER; note = thio; note = 2'-O-methyl
  modified_base 20: mod_base = OTHER; note = thio; note = 2'-fluoro
  modified_base 21: mod_base = OTHER; note = 2'-O-methyl
agtaggngagcactgagaatt

SEQ ID NO: 7 - RNA, 19 nt, synthetic construct
  source 1..19: mol_type = other RNA
  misc_feature 1..19: note = sense strand
  modified_base 1..2: mod_base = OTHER; note = 2'-O-methyl; note = thio
  modified_base 3..4: mod_base = OTHER; note = 2'-O-methyl
  modified_base 5: mod_base = OTHER; note = 2'-fluoro
  modified_base 6: mod_base = OTHER; note = 2'-O-methyl
  modified_base 7..9: mod_base = OTHER; note = 2'-fluoro
  modified_base 10..19: mod_base = OTHER; note = 2'-O-methyl
cagtgttcttgctctataa

SEQ ID NO: 8 - RNA, 21 nt, synthetic construct
  source 1..21: mol_type = other RNA
  misc_feature 1..21: note = antisense strand
  modified_base 1: mod_base = OTHER; note = 2'-O-methyl; note = thio
  modified_base 2: mod_base = OTHER; note = thio; note = 2'-fluoro
  modified_base 3..5: mod_base = OTHER; note = 2'-O-methyl
  modified_base 6: mod_base = OTHER; note = 2'-fluoro
  modified_base 7..13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17..18: mod_base = OTHER; note = 2'-O-methyl
  modified_base 19..20: mod_base = OTHER; note = 2'-O-methyl; note = thio
  modified_base 21: mod_base = OTHER; note = 2'-O-methyl
ttatagagcaagaacactgtt

SEQ ID NO: 9 - RNA, 21 nt, synthetic construct
  source 1..21: mol_type = other RNA
  misc_feature 1..21: note = antisense strand
  modified_base 1: mod_base = OTHER; note = 2'-O-methyl; note = thio
  modified_base 2: mod_base = OTHER; note = thio; note = 2'-fluoro
  modified_base 3..5: mod_base = OTHER; note = 2'-O-methyl
  modified_base 6: mod_base = OTHER; note = 2'-fluoro
  modified_base 7: mod_base = OTHER; note = n is ROR14-A
  modified_base 8..13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17..18: mod_base = OTHER; note = 2'-O-methyl
  modified_base 19..20: mod_base = OTHER; note = thio; note = 2'-O-methyl
  modified_base 21: mod_base = OTHER; note = 2'-O-methyl
ttatagngcaagaacactgtt